(19) United States
(12) Patent Application Publication
(10) Pub. No.: US 2016/0257927 A1
(43) Pub. Date: Sep. 8, 2016

(54) THREE-DIMENSIONAL CELL CULTURE USING NANOFIBER SLURRIES AND NANO-STRUCTURED SUBSTRATES

(71) Applicant: UCL BUSINESS PLC, London (GB)

(72) Inventors: Chaojie Luo, London (GB); Ivan Gout, Ruislip (GB); Ahmed Abdullah Ahmed, London (GB); Stoyan Smoukov, Cambridge (GB); Simeon Dobrev Stoyanov, Spijkenisse (NL)

(21) Appl. No.: 14/636,868

(22) Filed: Mar. 3, 2015

(51) Int. Cl. C12N 5/00

(52) U.S. Cl. CPC C12N 5/0068; C12N 2533/30; C12N 2513/00

(57) ABSTRACT

The present invention provides a method of culturing cells and/or controlling cell behaviour, which method comprises:
(a) introducing one or more cells into a medium; and
(b) allowing said one or more cells to grow within and/or interact with the medium,
   wherein said medium comprises a three dimensional homogeneous distribution of shear-spun nanofibers.

### TECHNICAL FIELD

The present invention relates to the use of shear-spun fibers as three-dimensional (3D) cell culture scaffolds, such as mats containing nanofibers and microfibers, or nanofiber slurries. In particular, the invention relates to the use of nonwovens containing short-length nanofibers and microfibers for regulating or promoting cell function or gene expression.

### BACKGROUND

Fibers form, in part or in whole, a large variety of both consumer and industrial materials. There are two main structural classes of fiber materials: woven and non-woven. Non-woven fiber materials tend to have lower production costs.

On a different note, in vitro cell cultures are used in preclinical testing in the medical field. For instance, they are used for preclinical anticancer drug screening prior to in vivo cell testing. In this regard, testing has generally been carried out by culturing cells on two-dimensional (2D) flat surfaces. However, such 2D models do not mimic an in vivo environment as they may lack e.g. the 3D functionality of cell-cell interactions. Thus, drug sensitivity of in vitro 2D cell cultures often differ significantly from that of in vivo cells, leading to inaccurate preclinical screening and poor predictions of drug efficacy in vivo.

To date, a number of 3D cell culture platforms have been developed with the aim of providing applications in e.g. tissue engineering, and regenerative medicine drug screening and development. Designing a 3D scaffold which effectively mimics the in vivo environment has proven not to be straight-forward, though (Comley, J (2010) “3D Cell Culture: easier said than done” Drug Discovery World).

In cancer research, the most well-known 3D cell models are cancer spheroids (<500 μm in diameter), grown for anti-cancer drug screening and tumor modeling in cell and molecular biology studies. However, the cancer spheroid model lacks e.g. stromal cells and extracellular matrix (ECM) and therefore fails to represent important regulatory interactions with the extracellular surroundings. Their lack of mechanical support also makes them hard to handle (Hutmacher, et al. (2009) J. Cell. Mol. Med. 13:1417).

The ECM is made of fibrous biopolymers, chiefly collagen. Thus, fibrous biopolymers have been shown to mimic the ECM and promote cell growth, e.g. cellulosic microfibers (˜100 μm) which promote cardiac cell growth, enhanced cell packing, connectivity and electromechanical functionality (Entcheva, et al. (2004) Biomaterials, 25:5753-5762; Semino, et al. (2003) Differentiation, 71:262-270). However, cell migration and the depth of cell growth in developed scaffolds is limited.

As regards efforts to mimic the ECM, a variety of scaffolds have been described, chiefly under two categories: animal-sourced de-cellularised collagen gel matrices, and animal-free synthetic polymer scaffolds.

Animal-based gel scaffolds of biopolymers derived from de-cellularised animal tissues, such as collagen hydrogels and Matrigel™, have been described for use as scaffolds for 3D cell culture (Kleinman, et al. (1982) Biochemistry 21:6188-6193; Bell and Ivarsson, (1979) Proc Natl Acad Sci, 76:1274-1278). Natural scaffolds may exhibit good biocompatibility as compared to synthetic materials. However, their clinical use can be complicated by potential disease transmission, the possibility of undesirable immune response, and ethical issues due to their animal-origin.

De-cellularized scaffolds of animal tissues have been also described, but present the following problems:

(1) porosity for cell-movement inside the scaffold: reconstituted natural ECM hydrogels are not porous immediately after de-cellularisation, and require additional techniques such as rapid freeze-drying to make random pores with limited pore-to-pore interconnectivity, which cause uneven cell distribution and suboptimal nutrient/waste diffusion;

(2) batch-to-batch variations due to animal-origin: this causes uncontrollable and undefined biomolecule-compositions from different batches of animal sources, making it difficult to compare cell culture and anticancer drug-screening results between scaffold batches;

(3) the high cost and animal-origin hamper affordable, large-scale applications and raise ethical questions; and

(4) it can be hard to tailor the breakdown rate of animal-derived scaffolds such as collagen, which rely on proteolysis by enzymes.

Synthetic scaffolds can overcome problems associated with animal-based scaffolds. To date, various synthetic cell culture substrates have been described, but they have had limited success. Current synthetic scaffolds are comprised mainly of macro- or micro-scale fibers constructed layer-by-layer using methods such as printing or weaving. Nanofiber layers in the scaffold are added alternatingly to this layered structure using methods such as electrospinning.

Polymeric nanofibers have been described for use in cell biology applications. Thus, the ability of electrospinning to generate nanofibers of topographical resemblance to the ECM collagen fibrils has been noted in the literature (Matthews, et al. (2002) Biomacromolecules 3:232). However, these nanofibers are generally thin, light and often hydrophobic. Consequently, synthetic nanofibers prepared by such techniques from hydrophobic polymers such as 100% PLA or polycaprolactone nanofibers, which may be supplied in the form of a free floating disc, encounter problems in that they are hard to moisten and tend to aggregate or float upwards in cell culture media due to air stuck to their surface. Many hydrophobic materials are made super-hydrophobic (water contact angles greater than 150 degrees) by the presence of nanoscale features. Without fixation onto a well-plate prior to cell culture, the nanofibers and nanoporous surfaces of the disc collapse under high surface tension of the aqueous media. Thus, commercial electrospun nanofibers for cell culture are generally fixed onto the surface of the substrate/bottom of well-plates using a bio-adhesive prior to cell seeding (see e.g. US Patent Application 20100190254). Such thin mats can provide nano-topography that improves cell-substrate interactions, but are not suitable as scaffolds for 3D cancer models and tissue/organ regeneration programs.

Electrospun nanofiber meshes have also been described. However, these are generally unable to effectively mimic in vivo environments as they are often <300 μm (more often <100 μm) in thickness, which may be equivalent to at most only a few layers of cells, depending on the cell type. Thicker electrospun nanofiber substrates (or mats) have been described too, but these do not support 3D cell growth as they are too dense, with pores too small for cells to penetrate (Kwon, et al. (2005) Biomaterials 26:3929). Moreover, as a general point, electrospinning is plagued by scale-up issues related to yield efficiency (Luo, et al. (2012) Chem. Soc. Rev. 41:4708; Place, et al. (2009) Nat. Mater. 8:457).

Other approaches for preparing 3D cell culture matrixes that have been described include strategies for making scaffolds containing a dual range of pore-sizes, which combine multiple techniques and/or stages of processing. In one instance, for example, to add surface micro- or nano-topography to macro-scale structures, larger macro-structures made by solid freeform fabrication or 3D printing are modified by a further process such as porogen leaching, laser irradiation, thermally induced phase separation, peptide self-assembly or nanofiber electrospinning (Zhong, et al. (2012) Tissue Eng. B 18:77; Kim and Kim. (2012) J. Mater. Chem. 22:16880; Pham, et al. (2006) Biomacromolecules 7:2796; US Patent Application 20100143435, 20110287082). However, the additional processing steps and combining multiple techniques are not straightforward.

The present invention aims to address the above-mentioned problems. In particular, the present invention aims to provide a new approach to culturing cells in vitro, which much more effectively mimics the in vivo extracellular environment, and in particular the nanofibrous topography as well as the microfibrous mechanical strength of the natural extracellular environment. The present invention also aims to achieve this using a medium which can be made via an easily reproducible and low-cost production process. As reported in the Examples section below, the methods and mediums of the present invention offer significant advantages over existing commercial cell culture models.

### SUMMARY

The present invention provides a method of culturing cells and/or controlling cell behaviour, which method comprises:

(a) introducing one or more cells into a medium; and

(b) allowing said one or more cells to grow within and/or interact with the medium,

wherein said medium comprises a three dimensional homogeneous distribution of shear-spun nanofibers.

In a preferred aspect, said method is a method of culturing cells and/or measuring cell proliferation. More preferably, said method is a method of culturing mammalian cells.

In another preferred aspect, said method is a method of regulating or promoting cell function in said one or more cells, or regulating or promoting gene expression in said one or more cells.

The present invention also provides a medium comprising a three dimensional homogeneous distribution of shear-spun nanofibers, which medium is suitable for use in culturing cells and/or controlling cell behaviour in a method of the invention as defined above. Preferably said medium is a solid scaffold comprising a three dimensional homogeneous distribution of microfibers and said nanofibers. In particularly preferred aspects the present invention provides (i) solid scaffolds in the form of dry thick discs (>100 μm thick) of interlocking homogeneously entangled microfibers and nanofibers, and (ii) wet homogeneous nanofiber slurries in liquid (typically aqueous) media.

The present invention also provides a process of producing a medium of the invention as defined above, which process comprises:

(a) wet laying a mixture of microfibers and said nanofibers, to form a nonwoven material;

(b) wet laying said nanofibers onto a woven or nonwoven material comprising microfibers; or

(c) wet laying said nanofibers onto themselves to form a nonwoven material.

The present invention also provides a medium as defined above, which comprises cultured cells. The present invention also provides a method of measuring cell proliferation, cytotoxicity and/or chemosensitivity in a sample of such cultured cells.

Other devices, apparatuses, systems, methods, features and advantages of the invention will be or will become apparent to one with skill in the art upon examination of the following figures and detailed description. It is intended that all such additional devices, apparatuses, systems, methods, features and advantages be included within this description, be within the scope of the invention, and be protected by the accompanying claims.

### BRIEF DESCRIPTION OF THE DRAWINGS

The invention can be better understood by referring to the following figures.

FIG. 1 is a Schematic diagram of nanofibers and microfibers wet-laid into a composite substrate.

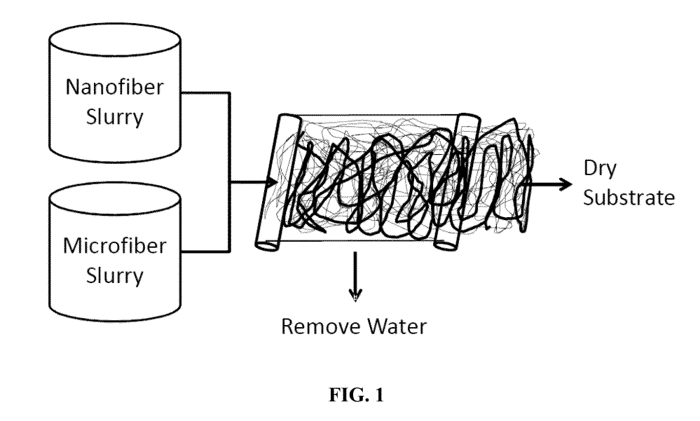

FIG. 2. SEM image showing a cross-sectional edge view of a wet laid mat comprised of 50% CA nanofibers (avg. diameter ˜400 nm) and 50% Rayon microfibers (avg. diameter 9 μm). The mat is 50 GSM and ˜200 micron in thickness.

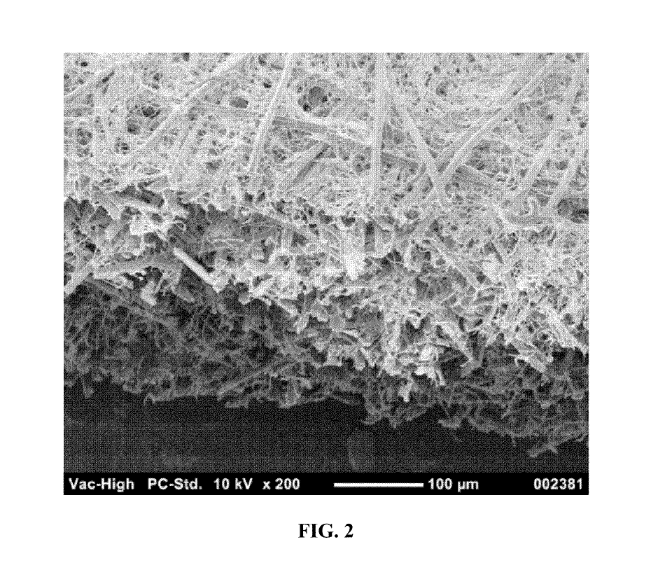

FIG. 3. SEM image showing a top view of a wet laid mat comprised of 50% PLA nanofibers (avg. diameter ˜400 nm) and 50% PET microfibers (avg. diameter 9 μm).

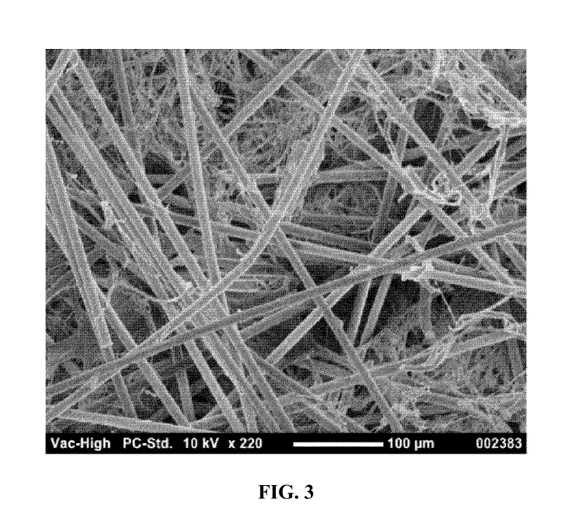

FIG. 4. Fluorescence and phase microscopy images of A549 human lung cancer (A), MCF-7 human breast cancer (B) and HEK293 human embryonic kidney cell lines (C) with stable expression of nuclear localized GFP (NLS-GFP).

FIG. 5. Comparative analysis of GFP-HEK293 cell growth in PLA/PET scaffold (comprised of 10% nanofiber and 90% microfiber), CA/PET scaffold (comprised of 30% nanofiber and 70% microfiber) and the commercial Nanofiber Solutions PCL (nsPCL) nanofiber scaffold. The fluorescent images at day 8 and 14 show that cells grow more efficiently and expand freely within shear-spun scaffolds when compared to nsPCL scaffold. In contrast to shear-spun scaffolds, cells grow mainly on the surface of the nsPCL scaffold with limited penetration of the nanofiber matrix. The scale bars indicate 130 μm.

FIG. 6. Analysis of the growth rate of GFP-HEK293 cells in nanofiber/microfiber scaffold comprised of 90% PET microfibers (avg. diameter 13 μm) and 10% PLA nanofibers (avg. diameter ˜700 nm). Cells were seeded at a low density of 5.8×10³ cells/cm². The formation of defined colonies from individual cells after 14 days in culture (A-D). Time-dependent growth of individual colonies of GFP-HEK293 cells (E).

FIG. 7. Analysis of the growth rate of GFP-MCF-7 and GFP-A549 cells in a scaffold comprised of 90% Rayon microfibers (avg. diameter 9 μm) and 10% CA nanofibers (avg. diameter ˜400 nm). Cells were seeded at a low density of (2.5×10³ cells/cm²) and cultured for 40 days. Time-dependent growth and fusion of two adjacent GFP-MCF-7 colonies (A) and time-dependent growth and scattering of single A549 cells (B). The scale bars indicate 130 μm (A-B).

FIG. 8. SEM image showing a top view of nanofiber/microfiber scaffold comprised of 90% PET microfibers (avg. diameter 13 μm) and 10% PLA nanofibers (avg. diameter ˜700 nm) (A). The pattern of growth of GFP-HEK293, GFP-MCF7 and GFP-A549 GFP-expressing in PLA/PET scaffold (B-D). Tested cells were seeded at a density of 500 cells/mm² and cultured for 20 days. The arrow indicates the microfibers junction, where a cluster of nanofibers is located (bird's nest). The scale bars indicate 100 μm (A) and 130 μm (B-D).

FIG. 9. SEM image showing a top view of nanofiber/microfiber scaffold comprised of 90% PET microfibers (avg. diameter 13 μm) and 10% PLA nanofibers (avg. diameter ˜700 nm) (A). The pattern of growth of EA.hy926 human umbilical vein endothelial, NIH3T3 mouse embryonic fibroblast and HFL1 human foetal lung fibroblast cell lines in PLA/PET scaffold (B-D). To visualize cells, they were stained with 1 μM Calcein-AM.

FIG. 10. SEM images showing a top view of PLA/PET scaffolds comprised of different ratios between PLA nanofibers (avg. diameter 700 nm) and PET microfibers (avg. diameter 13 μm), as indicated by the white box in the top right corner (A). The increase in the nanofiber content results in better cell attachment to the PLA/PET scaffolds (B).

FIG. 11. SEM images of CA/Rayon scaffolds (top view) comprised of different ratios between CA nanofibers (avg. diameter 400 nm) and Rayon microfibers (avg. diameter 9 jam), as indicated by white box in the top right corner. The increase in the CA nanofiber content results in better attachment of HEK293, MCF7 and A549 cells to the CA/Rayon scaffolds (B).

FIG. 12. Fluorescent and phase contrast images of GFP-HEK293 cells grown for 14 days in nanofiber/microfiber scaffold comprised of 90% PET microfibers (avg. diameter 13 μm) and 10% PLA nanofibers (avg. diameter ˜700 nm). Cryostat sections reveal that cells grow into 3D clusters, mainly at the microfibers cross junctions where nanofibers are located, indicating preferential cell attachment to nanofibers (as indicated in FIG. 8A)

FIG. 13. Fluorescent and contrast phase images of GFP-HEK293, GFP-MCF-7 and GFP-A549 cells grown for 14 days in nanofiber/microfiber scaffold comprised of 90% rayon microfibers (avg. diameter 9 μm) and 10% CA nanofibers (avg. diameter ˜400 nm). Cryostat sections reveal that HEK293 and MCF-7 cells grow into 3D cell clusters mainly at the microfibers cross-junctions, whereas A549 cells remain as single cell culture. The white arrows indicate 3D cell clusters (A-B) and single cells (C) and the scale bars indicate 35 μm (A-C).

FIG. 14. Testing cell viability of MCF-7, HFL1, EAhy926 and NIH3T3 cell lines grown for 20 days in nanofiber/microfiber scaffold comprised of 90% PET microfibers (avg. diameter 13 μm) and 10% PLA nanofibers (avg. diameter ˜700 nm) using the LIVE/DEAD viability/cytotoxicity Kit (Invitrogen). The fluorescent images show high cell viability of cultured cell lines in shear-spun scaffolds (indicated by Calcien-AM staining, top panel) and few dead cells (indicated by Ethidium homodimer-1 (EthD-1), bottom panel).

FIG. 15. Cross-cryosection images of GFP-HEK293 cells grown for 2 days in nanofiber/microfiber scaffolds: PLA/PET (A) and CA/Rayon (B) with varying nanofiber:microfiber ratios as indicated by the white boxes on the top right corner. The cryostat sections reveal that cells infiltrate and grow inside CA/Rayon and PLA/PET scaffolds up to 1:9 and 1:1 nanofiber:microfiber respectively. Beyond such ratios cells grow on the top of the scaffold. The cryosection analysis also shows CA/Rayon scaffolds remain compact and fibers are tightly entangled after cell culture, whereas the PLA/PET scaffolds have lower packing-integrity between fibers and disentanglement of fibers is evident after cell culture.

FIG. 16. Calibration graphs for MTS cell proliferation assay to convert the absorbance values to cell number in order to be able to compare cell proliferation data between 2D cell culture and 3D scaffolds (CA/Rayon or PLA/PET), provided that the same incubation time is applied. The graphs show the linear relationship between the absorbance at 490 nm of formazan and known cell number which clearly does not hold at higher cell number of A549 and MCF-7 cells.

FIG. 17. Comparative growth curve analysis of cancer cell lines in 2D and 3D cultures using the MTS CellTiter 96® Aqueous One Solution assay. GFP-HEK293, GFP-A549 and GFP-MCF-7 cell lines were seeded in 2D culture (A-B) and 3D nanofiber/microfiber scaffolds; PLA/PET, 3:7 (A) and CA/Rayon, 1:9 (B). The graphs demonstrate the significant difference in A549 and MCF-7 cell proliferation rate between 2D culture and 3D nanofiber/microfiber scaffolds, while HEK293 cell proliferation rate remain similar for both platforms.

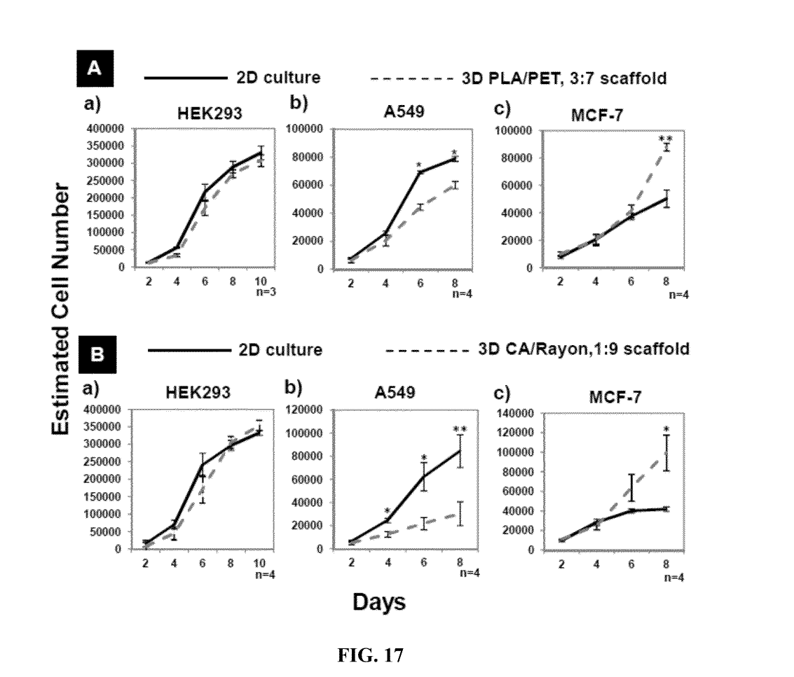

FIG. 18. Chemosensitivity of cancer cell lines in response to 48 hrs treatment with anticancer drugs when cultured in 2D Tissue Culture Plates (TCP) and 3D CA/Rayon, 1:9 scaffold. Lung cancer A549 cells were more sensitive to 5-fluorouracil and doxorubicin in 2D TCP than in 3D scaffolds (A) whereas breast cancer MCF-7 cells were more chemosensitive in 3D scaffold than in 2D TCP.

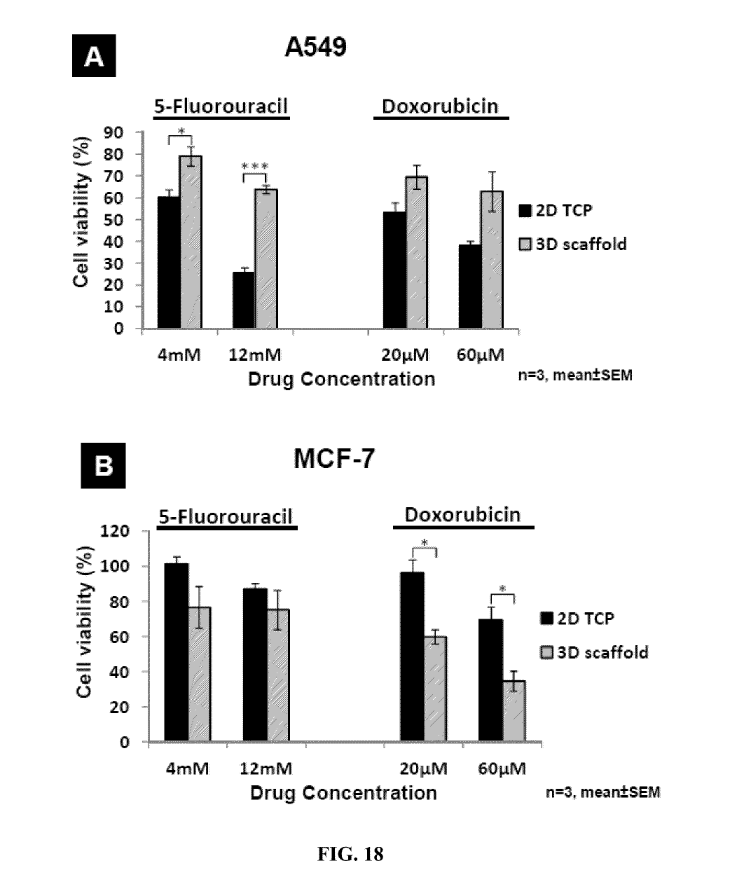

FIG. 19. Fluorescence, phase contrast and merged images of NIH3T3 mouse embryonic fibroblast (A), HFL1 human foetal lung fibroblast (B) and EA.hy926 human umbilical vein endothelial (C) with stable expression of red fluorescent protein (RFP). The images show the pattern of cell growth for the aforementioned cell lines when cultured in 2D culture plates. The scale bars indicate 65 μm.

FIG. 20. Fluorescent images of co-cultures (A and C) and tri-cultures (B and D) between cancer (A549 or MCF-7), fibroblast (NIH3T3) and/or endothelial (EA.hy926) cell lines when cultured for 12 days in a scaffold comprised of 90% rayon microfibers (avg. diameter 9 μm) and 10% CA nanofibers (avg. diameter ˜400 nm). The images show the efficient growth and cell cluster formation of different cell types when co-cultured or tri-cultured in shear-spun scaffold. The ratios indicate the cell number ratio between different cell types, the white arrows indicate cancer cell clusters, dashed arrows indicate fibroblast cells, circles indicate endothelial cells and the scale bars indicate 100 μm.

FIG. 21. Confocal z-stack images of monoculture (A), co-cultures (B and C) and tri-culture (D) of breast cancer (MCF-7), fibroblast (NIH3T3) and/or endothelial (EA.hy926) cell lines when cultured in a scaffold comprised of 90% rayon microfibers (avg. diameter 9 μm) and 10% CA nanofibers (avg. diameter ˜400 nm). The fluorescent images show the pattern of cell growth and the formed cell clusters of different cell types in mono-, co- and tri-cultures in shear-spun scaffold. The ratios indicate the cell number ratio between different cell types, the white arrows indicate cancer cell clusters, dashed arrows indicate fibroblast cells, circles indicate endothelial cells and the scale bars indicate 65 μm.

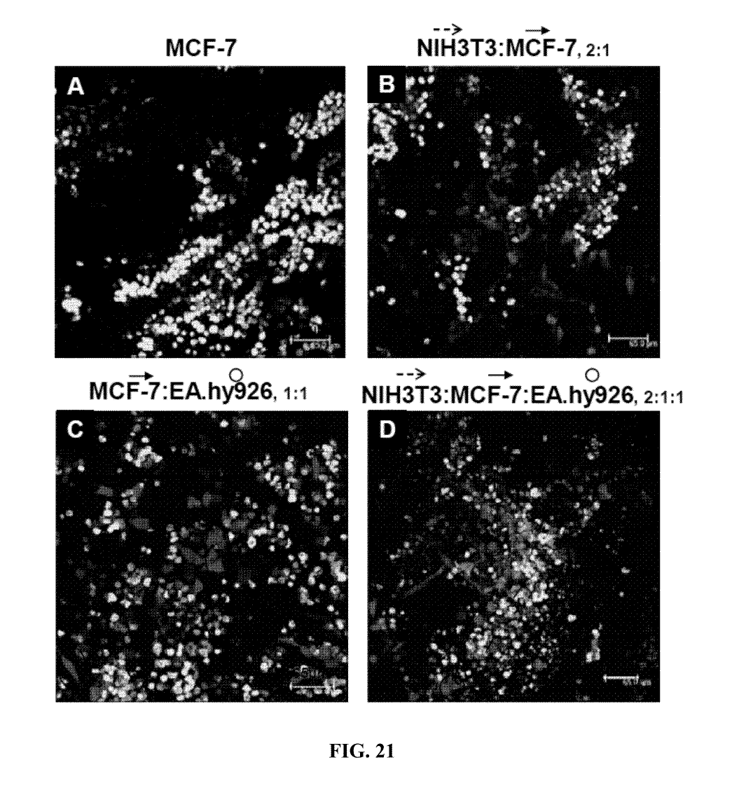

### DETAILED DESCRIPTION

The present invention provides a method of culturing cells and/or controlling cell behaviour, which method comprises:

(a) introducing one or more cells into a medium; and

(b) allowing said one or more cells to grow within and/or interact with the medium,

wherein said medium comprises a three dimensional homogeneous distribution of shear-spun nanofibers.

In this regard, said three dimensional homogeneous distribution of shear-spun nanofibers is intended to refer to an environment in which the shear-spun nanofibers are distributed homogenously in all three dimensions, i.e. akin to a single phase with no discontinuities in it. This is in contrast to e.g. a layered structure in which homogeneous distribution would be possible in two dimensions at most. In this regard, in terms of the scale over which the single phase homogeneous distribution is intended to extend, it preferably extends over a distance of at least around 50 μm in each dimension, such as at least 100 μm, at least 150 μm, at least 200 μm, at least 250 μm, at least 300 μm, at least 350 μm, at least 400 μm, at least 450 μm, at least 500 μm, at least 600 μm, at least 700 μm, at least 800 μm, at least 900 μm, or at least 1000 μm in each dimension.

The medium for use in the present invention comprises a three dimensional homogeneous distribution of shear-spun nanofibers. In this regard the reference to the fibers having been “shear spun” is intended to indicate that they have been obtained (or are obtainable) by a method which comprises introducing a polymer solution into a dispersion medium and shearing the polymer solution. Dispersed-phase components of the mixture may thereby be spun into elongated fibers. In a preferred aspect, such a process comprises:
(i) flowing a dispersion medium through a conduit;
(ii) introducing a fiber precursor solution (polymer solution) into the dispersion medium to form a dispersion system comprising the dispersion medium and a plurality of dispersed-phase components of the fiber precursor solution, wherein the fiber precursor solution comprises a polymer dissolved in a polymer solvent, and the dispersion medium comprises an anti-solvent; and
(iii) shearing the dispersed-phase components by flowing the dispersion system through the conduit, wherein a plurality of nanofibers are formed in the dispersion medium.

Suitable procedures for carrying out the process of this preferred aspect of the invention are described in WO 2013/172831, the content of which is incorporated herein in its entirety. The antisolvent used in the process of this preferred aspect of the invention may be thought of as a coagulant and causes the polymer to precipitate out of solution. The antisolvent should be sufficienctly miscible with the polymer solvent to enable the nanofiber formation. The polymer solution resides in the dispersion medium in the form of a dispersed phase comprising a plurality of dispersed-phase components that are dispersed throughout the volume of the dispersion medium. These components may be in the form of liquid streaks, liquid strands, and/or liquid droplets of varying shape. The polymer solution may enter the dispersion medium already in the form of dispersed-phase components or may enter in a continuous stream and break up into dispersed-phase components in the dispersion medium. In any case, once the polymer solution has been introduced into the dispersion medium (or during its introduction) the dispersion system is sheared (in a batch or continuous process), which deforms the dispersed-phase polymer solution into liquid filament streams due to capillary instabilities. The filaments are further stretched under a mechanism of shear-force elongation and the polymer solvent, being miscible with the dispersion medium, diffuses out from the dispersed-phase components/filaments into the dispersion medium. As a result, insoluble nanofibers composed of the polymer are formed.

The use of shear spun nanofibers in the present invention is important because such nanofibers can be used to prepare the medium of the present invention via a liquid-based process, which in turn enables the preparation of a medium in which the said shear-spun nanofibers are homogeneously distributed in three dimensions. Thus, the nanofibers can be easily mixed throughout the z-axis of a microfiber structure, much like native ECM. In contrast, the generation of polymer nanofibers as homogeneous suspensions has not been achieved by other fiber formation methods such as electrospinning, melt blowing and wet spinning. Indeed, nanofibers made by such techniques are generally formed as dry fibers, and so cannot be used to prepare a medium comprising a 3D homogeneous distribution of nanofibers.

Thus, the medium of the present invention is preferably made (or is obtainable by) wet-laying the said nanofibers. To this end, as mentioned above, in a preferred aspect the medium of the present invention is a solid scaffold made (or obtainable) by:
(a) wet laying a mixture of microfibers and said nanofibers, to form a nonwoven material;
(b) wet laying said nanofibers onto a woven or nonwoven material comprising microfibers; or
(c) wet laying said nanofibers onto themselves to form a nonwoven material.

Preferably the solid scaffold of the invention is made (or is obtainable) by approach (a) or (b) as set out above.

By way of example, in line with approach (a) polymeric nanofibers can also be wet laid together with other nano- or microfibers to form a nonwoven substrate containing many types of fibers; in line with approach (b) nanofibers can be wet laid deposited onto a non-woven or woven substrate, which is placed on a filter mesh, preferably of 27-200 microns pore size; and in line with approach (c) nanofibers can be deposited onto themselves without a substrate with basis weights ranging from 4 to 800 GSM or higher. In this case the length is important as longer length fibers provide mat integrity and strength.

By way of a further example, the medium may be made by a process of the type described in WO2014/116946, the content of which is incorporated herein in its entirety. In this regard it is noteworthy that WO2014/116946 contains no suggestion that the wet-laying process it describes could advantageously be used to prepare mediums for use in culturing cells and/or controlling cell behaviour (the only products mentioned in this publication are clothing, textiles, medical prostheses, construction materials, reinforcement materials, and bather, filtration and absorbent materials). It is believed that the idea of preparing a 3D cell culture medium using shear spun nanofibers, and in particular by adding polymeric nanofibers to or incorporating them into a substrate by wet laying technique has not previously been disclosed in the art. However, the present inventors have found that preparing the medium from shear spun nanofibers in this way enables the preparation of cell culture media with a surprisingly excellent ability to mimic the in vivo environment—as is evident from the results reported below in the Examples, the medium of the present invention represents a significant improvement over current commercial alternatives. In this regard, previous approaches to preparing 3D cell culture media based on nanofibers typically rely on long (>20 cm) dry nanofibers formed by electrospinning and meltblowing technologies. In contrast, as described below, the use of shear spun nanofibers in the present invention allows the production of polymeric nanofibers in a liquid based process, which enables various advantages as described herein.

As mentioned above, in a preferred aspect the medium of the present invention is a solid scaffold. More preferably, said solid scaffold comprises microfibers in addition to the said nanofibers. More preferably still, said solid scaffold comprises a homogenous distribution of both the microfibers and the nanofibers, and may be made (or be obtainable) by process (a) or (b) above. In this regard, the desired pore size of the solid scaffold can vary depending on the type of cell to be cultured, as cell size may vary from e.g. around 10 μm to around 150 μm. Thus, preferably the solid scaffolds of the present invention have a pore size which enables attachment and migration of the relevant cell type within the scaffold, such as a pore size of at least around 10 μm. For instance, the pore size may be e.g. at least around 20 μm, at least around 30 μm, at least around 40 μm, or at least around 50 μm. The pore size may be up to (or over), around 60 μm, around 70 μm, around 80 μm, around 90 μm, around 100 μm, around 110 μm, around 120 μm, around 130 μm, around 140 μm, around 150 μm, around 160 μm, around 170 μm, around 180 μm, around 190 μm, around 200 μm, around 250 μm, around 300 μm, around 350 μm, around 400 μm, around 450 μm, around 500 μm, around 600 μm, around 700 μm, around 800 μm, around 900 μm, or in some cases around 1000 μm. In this regard, the pore size will of course be dictated primarily by the structure of the microfibers present in the solid scaffold. In one aspect of the invention the pore size is from 2 μm to 500 μm. Pore size may be ascertained by SEM.

In one preferred aspect the medium of the invention comprises a fabric substrate of cotton, synthetic or blend fibers containing wet laid polymeric, staple nanofibers of short cut lengths. Preferred possible sizes for the staple nanofibers include ones listed above for the nanofibers in general. The staple polymeric nanofibers can be wet laid onto a fabric substrate of cotton, synthetic or blend fibers; or the nanofibers can be wet laid with other fibers to form a nonwoven mat; or the nanofibers can be wet laid onto themselves to form a nonwoven containing only nanofibers. Also, the nanofibers mixed with microfibers can be put into slurry form in cell culture medium. In the present invention, when the nanofibers are used in combination with microfibers, the nanofibers and microfibers can be directly supplied in any medium of physiological relevance; the medium can be any cell culture medium specific for a cell type to be seeded. The fiber suspension can be used either directly as a 3D matrix or dried to provide desirable thickness and porosity as an interlocking solid dry nano-microfiber matrix.

### The Nanofibers

As used herein, the term nanofiber refers generally to an elongated fiber structure having an average diameter of at least around 40 nm, preferably at least around 50 nm, such as at least around 60 nm, around 70 nm, around 80 nm, around 90 nm, around 100 nm, around 150 nm, or around 200 nm. The average diameter is preferably no more than around 10 μm, such as no more than around 5 μm or around 2 μm. Preferred ranges for the average diameter include around 100 nm to around 10 μm, such as around 200 nm to around 1 μm.

A particularly preferred average diameter is one of around 300 nm to around 500 nm, such as around 350 nm to around 450 nm, typically around about 400 nm—these types of diameter are particularly preferred for cellulose acetate (CA) nanofibers.

Another particularly preferred average diameter is one of around 600 nm to around 800 nm, such as around 650 nm to around 750 nm, typically around about 700 nm—these types of diameter are particularly preferred for polylactic acid (PLA) nanofibers.

Further preferred average diameters include the following, namely average diameters ranging from less than 50 nm-10 μm in some examples, in other examples ranging from less than 100 nm-10 μm, and in other examples ranging from 200 nm-10 μm. In further examples, the average diameter ranges from 40 nm-5 μm, 40 nm-2 μm, 50 nm-5 μm, 50 nm-2 μm, 100 nm-5 μm, 100 nm-2 μm, 200 nm-5 μm, or 200 nm-2 μm.

The “average” diameter may take into account not only that the diameters of individual nanofibers making up a plurality of nanofibers formed by implementing the presently disclosed method may vary somewhat, but also that the diameter of an individual nanofiber may not be uniform over its length in some implementations of the invention.

Average nanofiber diameter may be determined by standard methods such as scanning electron microscopy (SEM) and, if necessary, image analysis algorithms.

The average length of the nanofibers may range from around 100 nm to millions of nm. Preferably the average length of the nanofibers is at least around 500 nm, such as at least around 1 μm, around 5 μm, or around 10 μm. The average length may be up to (or over) around 50 μm, around 100 μm, around 500 μm, around 1 mm, around 5 mm, around 1 cm, around 5 cm, around 10 cm, around 50 cm, around 1 m, around 2 m or around 5 m. In a preferred aspect of the invention the nanofibers have an average length of from around 0.02 mm to around 2 m, such as around 0.1 mm to around 10 cm.

Average nanofiber length may be determined by standard methods such as electrostatic classification.

The average length of the nanofibers should be greater than the average diameter of the nanofibers. Thus, the aspect ratio (length:diameter) of the nanofibers is preferably X:1 wherein X is at least around 5, such as at least around 10, around 50, or around 100. The value of X may be up to (or over) around 500, around 1,000, around 5,000, around 10,000, around 50,000, around 100,000, around 500,000, around 1,000,000, or sometimes even greater. Preferred values for X are from around 20 to around 200,000, such as around 200 to around 20,000. Fiber aspect ratios may be controlled during fiber production.

The nanofibers for use in the invention generally comprise one or more polymers as the predominant component. In principle, polymers for use in preparing the nanofibers may be any naturally-occurring or synthetic polymers capable of being fabricated into nanofibers, although some may be more preferred than others in certain contexts, e.g. depending on the type(s) of cell intended to be cultured. In a preferred embodiment the nanofibers may consist essentially of one or more polymers. As noted below, though, in other embodiments the nanofibers may include further components such as inorganic components, which may be present in the form of inorganic fibrils.

In a preferred aspect, the nanofibers for use in the invention comprise nanofibers of one or more polymers selected from the group consisting of a polyolefin, a polystyrene, a polycaprolactone, a polyacrylonitrile, a polyvinylidenedifluoride, a poly(vinyl chloride), a polytetrafluoroethylene, a poly(α-methylstyrene), a poly(acrylic acid), a poly(isobutylene), a poly(acrylonitrile), a poly(methacrylic acid), a poly(methyl methacrylate), a poly(l-pentene), a poly(1,3-butadiene), a poly(vinyl acetate), a poly(2-vinyl pyridine), a 1,4-polyisoprene, a 3,4-polychloroprene, a poly(ethylene oxide), a polyformaldehyde, a polyacetaldehyde, a poly(3-propionate), a poly(10-decanoate), a poly(ethylene terephthalate), a polycaprolactam, a poly(11-undecanoamide), a poly(hexamethylenesebacamide), a poly(m-phenylene terephthalate), a poly(tetramethylene-m-benzenesulfonamide), a polyacetal, a polyetheretherketone, a polyetherimide, a polyamide, a polyurea, a polyamideimide, a polyarylate, a polybenzimidazole, a polyester, a polycarbonate, a polyurethane, a polyimide, a polyhydrazide, a phenolic resin, a polysilane, a polysiloxane, a polycarbodiimide, a polyimine, an azo polymer, a polysulfide, and a polysulfone.

In a more preferred aspect, the nanofibers comprise nanofibers of one or more natural materials selected from the group consisting of cellulose, cellulose acetate (CA), PLA, silk, chitin, hemp and cotton. CA and PLA are particularly preferred. In one aspect the natural material is selected from the group consisting of cellulose, PLA, silk, chitin, hemp and cotton.

In another preferred aspect of the invention, polymers for use in preparing the nanofibers include high molecular weight (MW) solution-processable polymers such as polyethylene (more generally, various polyolefins), polystyrene, cellulose, cellulose acetate, poly(L-lactic acid) (PLA), polyacrylonitrile (PAN), polyvinylidenedifluoride (PVDF), conjugated organic semiconducting and conducting polymers, biopolymers such as polynucleotides (DNA) and polypeptides, etc.

Other examples of suitable polymers for forming the nanofibers include vinyl polymers such as, but not limited to, cellulose acetate propionate, cellulose acetate butyrate, polyethylene, polypropylene, poly(vinyl chloride), polystyrene, polytetrafluoroethylene, poly(α-methylstyrene), poly(acrylic acid), poly(isobutylene), poly(acrylonitrile), poly(methacrylic acid), poly(methyl methacrylate), poly(1-pentene), poly(1,3-butadiene), poly(vinyl acetate), poly(2-vinyl pyridine), 1,4-polyisoprene, and 3,4-polychloroprene. Additional examples include nonvinyl polymers such as, but not limited to, poly(ethylene oxide), polyformaldehyde, polyacetaldehyde, poly(3-propionate), poly(10-decanoate), poly(ethylene terephthalate), polycaprolactam, poly(l 1-undecanoamide), poly(hexamethylenesebacamide), poly(m-phenyleneterephthalate), poly(tetramethylene-m-benzenesulfonamide). Additional polymers include those falling within one of the following polymer classes: polyolefin, polyether (including all epoxy resins, polyacetal, polyetheretherketone, polyetherimide, and poly(phenylene oxide)), polyamide (including polyureas), polyamideimide, polyarylate, polybenzimidazole, polyester (including polycarbonates), polyurethane, polyimide, polyhydrazide, phenolic resins, polysilane, polysiloxane, polycarbodiimide, polyimine, azo polymers, polysulfide, and polysulfone.

The polymer(s) used to form the nanofibers can be synthetic and/or naturally-occurring. Examples of natural polymers include, but are not limited to, polysaccharides and derivatives thereof such as cellulosic polymers (e.g., cellulose and derivatives thereof as well as cellulose production by-products such as lignin) and starch polymers (as well as other branched or non-linear polymers, either naturally occurring or synthetic). Exemplary derivatives of starch and cellulose include various esters, ethers, and graft copolymers. The polymer may be cross-linkable in the presence of a multifunctional cross-linking agent or cross-linkable upon exposure to actinic radiation or other type of radiation. The polymer may be a homopolymer of any of the foregoing polymers, a random copolymer, block copolymer, alternating copolymer, random tripolymer, block tripolymer, alternating tripolymer, or a derivative thereof (e.g. a graft copolymer, ester, or ether thereof), and the like.

Nanofibers for use in the invention should of course be biocompatible with the cells of interest. In one preferred aspect, the nanofibers are also biodegradable. In some aspects, preference may also be given to materials that do not trigger immune responses or the development of a fibrous capsule (scar-tissue). A degradable scaffold that gradually becomes replaced by the natural ECM secreted by the growing cells may be desirable for clinical purposes. Advantages of the invention include the facts that it provides scaffolds with quantifiable compositions, controllable rates of degradation, and economical plus scalable production.

The present invention enjoys the advantages associated with nanofibers, which may attain a high surface area comparable with the finest nanoparticle powders, yet are fairly flexible, and retain one macroscopic dimension, which makes them easy to handle, orient and organize. A further advantage is that the in vivo breakdown rate of synthetic biomaterials is tunable (unlike animal-derived scaffold materials). Polymers such as polyglycolide (PGA) undergo hydrolysis in situ. Consequently, for in vivo applications the body must be able to metabolize the monomeric products released during breakdown without a toxic or inflammatory response. For systems such as copolymers of PGA and polylactide (PLA), degradation rates can be readily tuned by the composition of PGA versus PLA, where a higher PGA content degrades faster. A further advantage of these materials is that they can be made to closely represent the size and dimensions of natural polymeric fibers found in native ECM.

In a preferred aspect of the invention the nanofibers present in the medium may be uniform in terms of length and/or diameter and/or aspect ratio and/or composition. Alternatively, a mixture of two or more different types may be present.

### The Microfibers

In a preferred aspect, said medium of the invention comprises a three dimensional homogeneous distribution of both microfibers and said nanofibers. In other words, the medium containing a 3D homogeneous distribution of nanofibers also includes a 3D homogeneous distribution of microfibers. More preferably in this regard the medium is a solid scaffold. The microfibers may be present in the form of a woven or nonwoven material.

In a preferred aspect of the invention, the microfibers have an average diameter of at least around 0.5 μm, preferably at least around 1 μm, such as at least around 2 μm, around 3 μm, around 4 μm, around 5 μm, around 6 μm, or around 8 μm. The average diameter is preferably no more than around 100 μm, such as no more than around 50 μm, around 40 μm, around 30 μm, around 20 μm, or around 15 μm. A particularly preferred average diameter is one of around 5 μm to around 20 μm, such as (i) around 8 μm to around 10 μm (typically around about 9 μm), or (ii) around 12 μm to around 14 μm (typically around about 13 μm). These types of diameter are particularly preferred for Rayon and/or PET microfibers.

Average microfiber diameter may be determined by standard methods such as scanning electron microscopy (SEM) and, if necessary, image analysis algorithms.

The average length of the microfibers is preferably at least around 10 μm, such as at least around 100 μm or around 500 μm. The average length may be up to (or over) around 1 mm, around 5 mm, around 1 cm, around 5 cm, around 10 cm, 50 cm, around 1 m, around 2 m or around 5m.

Average microfiber length may be determined by standard methods such as electrostatic classification.

The average length of the microfibers should be greater than the average diameter of the microfibers. The aspect ratio (length:diameter) of the microfibers is preferably X:1 wherein X is at least around 5, such as at least around 10, around 50, or around 100. The value of X may be up to (or over) around 500, around 1,000, around 5,000, around 10,000, around 50,000, around 100,000, around 500,000, around 1,000,000, or sometimes even greater. Preferred values for X are from around 20 to around 200,000, such as around 200 to around 20,000. Fiber aspect ratios may be controlled during fiber production.

The microfibers for use in the invention generally comprise one or more polymers as their predominant component. The microfibers may comprise natural (e.g. cotton), synthetic or blend fibers. In the context of the present invention a wide variety of polymers and fabric substrates of cotton, synthetic or blend fibers may be utilized as starting materials for producing 3D scaffolds for mammalian cell culturing, examples of which are described herein.

In a preferred aspect, the microfibers comprise microfibers of one or more selected from the group consisting of cotton, cellulose, Lyocell, acetate, cellulose acetate, rayon, silk, wool, hemp, spandex, polyolefin, polyamide, aramid, acrylic, polyester, polyurethane, glass microfibers, and fibreglass.

In the context of the present invention, a nonwoven or woven fabric substrate or web comprising microfibers can be made from natural or synthetic fabrics and may be composed of fibers of cotton, cellulose, Lyocell, acetate, cellulose acetate, rayon, silk, wool, hemp, spandex (including LYCRA), polyolefins (polypropylene, polyethylene, etc.), polyamide (nylon 6, nylon 6-6, etc.), aramids (e.g. Kevlar®, Twaron®, Nomex, etc.), acrylic, or polyester (polyethylene teraphthalate, trimethyleneterephthalate), polyurethane, glass microfibers, fibreglass. Further options include fabric blends, i.e. fabrics of two or more types of fiber. Typically these blends are a combination of a natural fiber and a synthetic fiber, but can also include a blend of two natural fibers or two synthetic fibers. In this regard, “web” is intended to refer to a fibrous material capable of being wound into a roll, and a nonwoven web is intended to refer to a web of individual fibers or filaments which are interlaid and positioned in a random manner to form a planar material without identifiable pattern, as opposed to a knitted or woven fabric. Nonwoven webs have been in the past formed by a variety of processes known to those skilled in the art. For example, they can be meltblown, spunbound, wet-laid, dry-laid, or prepared by bonded carded web processes.

Microfibers for use in the invention should of course be biocompatible with the cells of interest. In one preferred aspect, the microfibers are also biodegradable. In some aspects, preference may also be given to materials that do not trigger immune responses or the development of a fibrous capsule (scar-tissue). A degradable scaffold that gradually becomes replaced by the natural ECM secreted by the growing cells may be desirable for clinical purposes. As noted above, an advantage of the invention is that it provides scaffolds with quantifiable compositions, controllable rates of degradation, and economical plus scalable production.

In a preferred aspect of the invention the microfibers present in the medium may be uniform in terms of length and/or diameter and/or aspect ratio and/or composition. Alternatively, a mixture of two or more different types may be present.

### Further Possible Components of the Medium

The medium of the present invention may further comprise one or more fibrils. In this regard the term fibril refers generally to an elongated fiber structure having an average diameter ranging from about 1 nm-1,000 nm in some examples, in other examples ranging from about 1 nm-500 nm, and in other examples ranging from about 25 nm-250 nm. Fibrils may be formed by phase separation from nanofibers. In these methods, the diameter of a fibril is generally smaller than the diameter of the nanofiber with which it is associated, and typically smaller by an order of magnitude. A fibril may be composed of an inorganic precursor or an inorganic compound. Fibrils may also be characterized as nanofibers. In the present disclosure, the term “fibrils” distinguishes these structures from the polymer nanofibers that may be utilized to form the inorganic fibrils. The length of the fibrils may be about same as the polymer nanofibers or may be less.

Such inorganic fibrils may be present in a medium of the invention as part of the nanofibers for use in the invention, and in particular in the form of composite inorganic/polymer nanofibers. Alternatively, they may be included separately. In either case, though, the fibrils are preferably formed by combining a relevant inorganic precursor with the nanofiber precursor (polymer solution) prior to the shear spinning that is carried out in order to prepare the shear-spun nanofiber for use in the invention. In this regard, as noted above, the medium for use in the present invention comprises a three dimensional homogeneous distribution of shear-spun nanofibers, and the reference to the fibers having been “shear spun” is intended to indicate that they have been obtained (or are obtainable) by a method which comprises introducing a polymer solution into a dispersion medium and shearing the polymer solution. Dispersed-phase components of the mixture may thereby be spun into elongated fibers. To this end, if it is desired to include one or more further additives in the medium of the invention in this way (including in particular those described in the preceding paragraph), then the additive(s) may preferably be combined with the nanofiber precursor (polymer solution) prior to shear spinning so as to form an inorganic fibril. Possible methods for effecting this are described in US20120309250, the content of which is incorporated herein in its entirety, and include the following methods (i) and (ii).

Method (i) is a method for fabricating composite inorganic/polymer nanofibers and the method comprises:
(i) introducing a mixture of a polymer solution (nanofiber precursor) and an inorganic precursor into a dispersion medium to form a dispersion system comprising the dispersion medium and a plurality of dispersed-phase components of the mixture, wherein the polymer solution comprises a polymer dissolved in a polymer solvent, and the dispersion medium comprises an anti-solvent for the polymer such that the polymer solvent is miscible with the anti-solvent;
(ii) forming a plurality of composite nanofibers by shearing the dispersed-phase components, wherein phase separation occurs between the polymer and the inorganic precursor such that a plurality of inorganic fibrils are formed in each nanofiber; and
(iii) forming an inorganic compound from the inorganic precursor, wherein the inorganic fibrils comprise the inorganic compound.

Method (ii) is a method for fabricating inorganic fibrils and the method comprises:
(i) introducing a mixture of a polymer solution (nanofiber precursor) and an inorganic precursor into a dispersion medium to form a dispersion system comprising the dispersion medium and a plurality of dispersed-phase components of the mixture, wherein the polymer solution comprises a polymer dissolved in a polymer solvent, and the dispersion medium comprises an anti-solvent for the polymer such that the polymer solvent is miscible with the anti-solvent;
(ii) forming a plurality of composite nanofibers by shearing the dispersed-phase components, wherein phase separation occurs between the polymer and the inorganic precursor such that a plurality of inorganic fibrils are formed in each nanofiber;
(iii) forming an inorganic compound from the inorganic precursor, wherein the inorganic fibrils comprise the inorganic compound; and
(iv) removing the polymer from the inorganic fibrils.

In a preferred aspect of method (i), the inorganic precursor is selected from the group consisting of titania precursors, silica precursors, alumina precursors, zirconia precursors, bioceramic precursors, bioactive glass precursors, methodoxides, ethoxides, sec-butoxides, and combinations of any two or more thereof.

In another preferred aspect of method (i), the inorganic precursor comprises a hydrolysable metal compound.

In another preferred aspect of method (i), the method comprises introducing an additive to the dispersion medium wherein the composite nanofibers comprise the polymer, the inorganic fibrils, and the additive retained by the polymer, and wherein said introducing occurs at a time selected from the group consisting of: before introducing the mixture into the dispersion medium, while introducing the mixture into the dispersion medium, after introducing the mixture into the dispersion medium, and combinations of any two or more thereof.

In a preferred aspect the medium of the invention may comprise one or more further substances selected from the group consisting of ceramics such as titania, alumina, zirconia and various clays, silica, glasses, bioceramics, bioactive glasses, metals (e.g., silver, gold, etc.), metal alloys, metal oxides, metalloids (e.g., silicon, germanium, semiconductor and quantum dot forming materials etc.) and their oxides, graphite, carbon black, various graphene nanosheets and carbon nanotubes (CNTs). Such additives are preferably combined with the nanofiber precurosor (polymer solution) prior to shear spinning in the manner described above, and may be included for various purposes such as imparting or enhancing a property or function of the nanofiber, for example strength, anti-bacterial activity, therapeutic activity (e.g., pharmaceutical drug crystals), conductivity, semiconductivity (e.g., quantum dots, semiconductor nanoparticles), magnetic behavior, porosity, hydrophobicity, selective permeability, selective affinity to various materials, adhesiveness, enzymatic or catalytic activity, biocompatibility, biodegradability, biological adhesion, biological recognition and/or binding, chemical inertness, polarity, selective retention and/or enrichment of analytes in analytical separation techniques. As one example, high molecular-weight polyethylene, known for its strength, could be strengthened by the incorporation of CNTs. Other types of additives that may be combined with the nanofiber precursor (polymer solution) prior to shear spinning include colorants (e.g. fluorescent dyes and pigments), odorants, deodorants, plasticizers, impact modifiers, fillers, nucleating agents, lubricants, surfactants, wetting agents, flame retardants, ultraviolet light stabilizers, antioxidants, biocides, thickening agents, heat stabilizers, defoaming agents, blowing agents, emulsifiers, cross-linking agents, waxes, particulates, flow promoters, and other materials added to enhance processability or end-use properties of the polymeric components.

In a preferred aspect the medium of the invention may comprise one or more further substances selected from the group consisting of a growth factor, anti-oxidant, differentiation inducer, hormone, vitamin, nucleic acid, drug, humectant, emollient, peptide, conditioner and cosmetic.

### Further Preferred Aspects of the Invention

In a preferred aspect, the medium of the invention comprises a three dimensional homogeneous distribution of microfibers and nanofibers (the nanofibers preferably being shear spun). More preferably in this regard the medium is a solid scaffold, which more preferably still is made (or obtainable) by (a) wet laying a mixture of said microfibers and nanofibers to form a nonwoven material, or (b) wet laying said nanofibers onto a woven or nonwoven material comprising said microfibers. The solid scaffold may preferably be a disc with a thickness of around 100 μm or more, such as e.g. around 150 μm or more, around 200 μm or more, around 250 μm or more, around 300 μm or more, around 350 μm or more, around 400 μm or more, around 450 μm or more, or around 500 μm or more.

In another embodiment of the invention, the nanofiber suspension obtained from the shear-spinning process used to form the nanofibers can be dried and supplied as shear-spun dry mats. The shear-spun dry mat typically consists essentially of an interlocking/entangled nanofiber-microfiber scaffold. The shear-spun nanofiber dry mats can be of any thickness from micrometer to centimeter-scale, although preferred possible thicknesses are mentioned above. The fiber aspect ratios in the dry scaffold can be controlled.

The medium of the invention can comprise (or consist of) uniform nanofibers or a mixture of various required fiber diameters. The proportion of nanofibers versus microfibers in the solid scaffold can be tailored to suit various porosity requirements for the proliferation of various cell type(s) inside the scaffold. Different cell types can have different cell sizes ranging from e.g. approximately 10 to 150 μm. When the medium is a fiber mat, said mat can be pre-placed at the bottom of a well plate in a retrievable-manner or as discs to be placed by the end-user.

The present invention seeks to mimic in vivo tissue architecture by allowing the utilisation of both micron length scales and nanometer scales in order to reproduce tissue. Microstructural features can provide for structural support (for regenerated tissue) and cell movement within the scaffold. For example, micro-scale architectures can help cells to infiltrate the internal 3D geometry by means of passive diffusion of cell suspensions, active cell migration, and maintenance of cell function. Meanwhile, the nanofibers facilitate cell attachment and signalling in the medium, which can help modulate cell differentiation, interaction and adhesion in response to nano-topography, mimicking native collagen fibrils and the ECM. Thus, tissue growth requires cell attachment and interactions between cell integrin receptors and adhesive ligands. This occurs in native tissue when inter-ligand distances are within the nanometer-scale. To this end, when the medium of the invention is a solid scaffold, the pores in the scaffold should be larger than the cells to be cultured. Preferred pore sizes are noted above. It is worth noting in this regard that the medium of the invention is preferably a three dimensional homogeneous distribution of microfibers and the said nanofibers. This is advantageous because the presence of just nanoscale features (alone) can lead to scaffolds that are not easily infiltrated if the cells are bigger than the pores. Pore size in fiber scaffolds is generally dependent on fiber diameter and packing density and so can be tailored depending on what type of environment is desired—e.g. a higher nanofiber content may lead to a lower pore size and vice versa.

When the medium of the invention comprises both microfibers and nanofibers, the weight ratio of nanofibers:microfibers is preferably from around 1:100 to around 100:1, such around 1:50 to around 50:1 or around 1:20 to around 20:1. More preferred possible ratios include ratios of around 9:1, around 7:3, around 1:1, around 3:7, and around 1:9, although the preferred ratio may depend on the identity of the nanofibers and the microfibers and the cell/tissue type(s) of interest.

Thus, in a preferred aspect, the medium of the invention comprises a three dimensional homogeneous distribution of microfibers and nanofibers, said microfibers are microfibers of polyethylene terephthalate (PET), said nanofibers are nanofibers of polylactic acid (PLA), and the weight ratio of said fibers of PET to said fibers of PLA is from 7:3 to 1:1. This can provide optimised cell attachment and growth. Preferably the medium is a solid scaffold as described herein.

In another preferred aspect, the medium of the invention comprises a three dimensional homogeneous distribution of microfibers and nanofibers, said microfibers are microfibers of rayon, said nanofibers are nanofibers of cellulose acetate (CA), and the weight ratio of said fibers of rayon to fibers of CA is from 9:1 to 7:3. Again, this can provide optimised cell attachment and growth. Preferably the medium is a solid scaffold as described herein.

In another preferred aspect the medium of the invention is a dispersion of the said nanofibers in a liquid. Said liquid is suitably a biologically compatible liquid and may preferably be selected from the group consisting of cell culture media, PBS and water. Typically it is an aqueous medium. In this regard, said nanofibers are preferably present in the liquid at a concentration whereby the nanofibers account for 0.1% to 10% by weight, preferably 0.1% to 5%, more preferably 0.1% to 2% by weight, yet more preferably 0.2% to 0.6% by weight of the dispersion. The dispersion may also comprise microfibers as described herein. If present, the microfibers may account for 0.1% to 20% by weight, preferably 0.1% to 10%, more preferably 0.1% to 5% by weight, yet more preferably 0.2% to 1% by weight of the dispersion.

In a preferred aspect of the invention, the aqueous medium can be a physiological saline medium or a cell culture medium for scalable and efficient cell growth, proliferation and differentiation in developed 3D nano/microfiber scaffolds.

In another embodiment of the invention, the dispersion (or suspension) can be mixed with other required additives in the form of particles, liquids and gels, to tailor for different structural and nutritional requirements for the growth of different cell types. The fiber suspension can be concentrated or diluted by adding or removing additional aqueous medium.

In one embodiment of the invention the 3D scaffolds are supplied as a suspension or nanofiber slurry—an aqueous product in a physiological medium or cell culture medium.

In a preferred aspect of the invention the fibers are directly produced in an aqueous medium as a homogeneous fiber suspension, preferably for subsequent cell seeding.

### Advantages and Uses of the Invention

The medium of the present invention is believed to provide a significantly improved in vitro representation of in vivo tissue self-assembly, as driven by active cell-cell and cell-extracellular matrix interactions, compared to previous cell culture models. The balance of these interactions can control normal morphogenesis, growth, proliferation and differentiation in healthy tissues, and its dysregulation is particularly prominent in cancer invasion and metastasis. The present invention can thus be used to analyse these phenomena. In particular, the present invention provides an improved tool for studying cell culture growth in 3D, and can enable straightforward processing with hydrophilic cell cultures in 3D.

The method of the invention may advantageously be used to investigate the optimal nanofiber:microfiber ratio for efficient attachment, culturing, etc of a given cell. Thus, the medium of the invention can be tailored to control desired features across the nanoscale, microscale, and macroscale in order to optimise the environment depending on the types of cell(s) and tissue(s) of interest to the study. For instance, the proportion of nanofibers versus microfibers in the suspension can be controlled and changed to any requirement.

The present invention enables methods for culturing a diverse range of mammalian cells. Preferably in this regard the medium of the invention is a solid scaffold made of staple polymer nanofibers and microfibers.

The present invention may be used to assist histological investigations. For instance, the invention provides methods for preparing cryosections of nanofiber/microfiber scaffolds with cultured cells for histological investigations.

As further illustrative possible uses of the medium of the present invention, it is compatible with cell-based assays for measuring cell proliferation, cytotoxicity and chemosensitivity; it can be used to investigate the differential cell proliferation rate of cancer cells for 3D nanofiber/microfiber scaffolds as compared to 2D tissue culture plates; it can be used to investigate differences in cell response to anticancer drugs between the environments of 3D nanofiber/microfiber scaffolds and 2D tissue culture plates; and it can be used to investigate cell infiltration inside the 3D environment of the nanofiber/microfiber scaffolds of the invention.

The present invention provides for the first time a fast, low-cost and reproducible process to produce 3D scaffolds for mono-, co-, and tri-culture of mammalian cells, that also allows nanofibers to be easily mixed with microfibers.

The medium and method of the present invention offer significant advantages over existing commercial cell culture models and methods. For instance, as reported below in the Examples, significant differences in cancer cell response to anticancer drugs are seen when comparing (a) the shear-spun 3D scaffold cultures obtained in accordance with the present invention, with (b) 2D cell cultures.

### EXAMPLES

The present application is further described by means of the examples, presented below. The use of these examples in no way limits the scope and meaning of the invention and is illustrative only. Likewise, this application is not limited to any particular preferred embodiments described herein.

Wet Laying Process:

Cellulose acetate (Eastman CA-398-10) nanofibers (average diameter of 400 nm and lengths of ˜200-700 mm or 2-10 mm) were mixed with 1.5 denier, 6 mm length PET microfibers (average diameter 9 μm) and added to water in a 10 inch×10 inch handsheet former. Water is removed by gravity through a 100 mesh stainless steel woven screen at a final basis weight of 50 grams per square meter. The sample is dried on an Emerson Apparatus Speed Dryer at 200 degrees Fahrenheit.

Generation of Stable Cell Lines Expressing Nuclear Localized GFP (NLS-GFP):

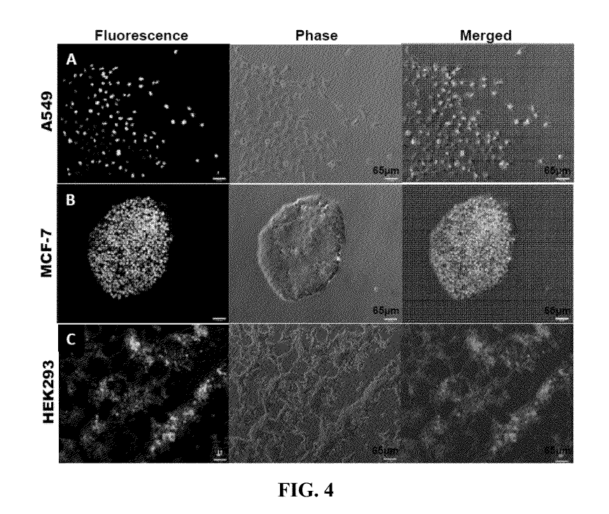

To aid visualization of cells in tested scaffolds, a panel of stable cell lines expressing NLS-GFP was generated. Generation of stable cell lines was achieved using the lentiviral protein expression system. Human embryonic kidney (HEK293), human breast cancer (MCF-7) and human lung cancer (A549) cell lines were infected with recombinant lentiviruses to direct the expression of green fluorescent protein fused to the nuclear localization sequence (NLS-GFP). Then, stably transduced cells were selected with 1.0 μg/mL puromycin for 2-3 weeks. FIG. 4A-C shows fluorescent and phase contrast images of A549 (A), MCF-7 (B) and HEK293 (C) with stable expression of nuclear localized GFP. Notably, selected cell lines exhibit different patterns of growth when cultured in 2D tissue culture plates.

Comparative Analysis of GFP-HEK293 Cell Growth in Wet-Laid Nanofiber/Microfiber and Commercial Scaffolds:

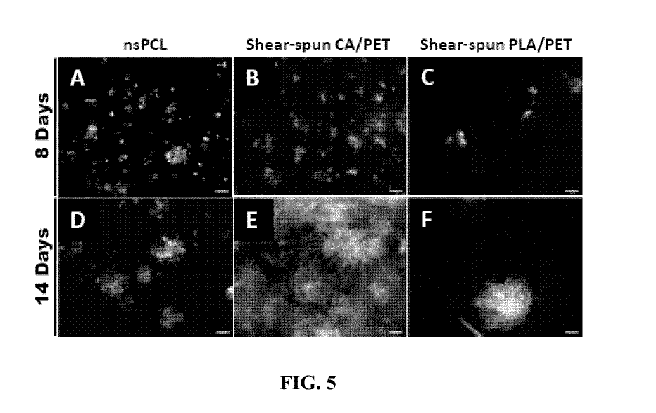

The growth of GFP-HEK293 cells in nanofiber/microfiber scaffolds was compared to that in a commercial Nanofiber Solutions electrospun PCL (nsPCL) scaffold. The thickness of the nsPCL scaffold is ˜20 μm compared to ˜0.5-1 mm of all tested nanofiber/microfiber scaffolds. The nsPCL scaffold is comprised of tightly packed polycaprolactone nanofibers. GFP-HEK293 cells were seeded at a density of 3.3×10⁴ cells/cm² on PLA/PET (10% PLA nanofiber and 90% PET microfiber, 1:9 ratio), CA/PET (30% nanoCA:70% microPET, 3:7 ratio) and nsPCL scaffolds. Culturing cells for two weeks revealed that GFP-HEK293 cells grow more efficiently within these scaffolds when compared to nsPCL scaffold. GFP-HEK293 cells are able to expand freely inside the nanofiber/microfiber scaffolds but not in nsPCL scaffold. Detailed examination of cell growth in these scaffolds showed that GFP-HEK293 cells grow mainly on the surface of the nsPCL scaffold with limited penetration of the nanofiber matrix. It is most likely that cultured cells are not able penetrate a dense mesh of nanofibers and therefore grow on the top of the scaffolds. In contrast, extensive growth of GFP-HEK293 cells was observed within PLA/PET and CA/PET scaffolds, where cells formed large 3D clusters or individual colonies (FIG. 5).

Time-Dependent Growth of Cancer Cell Lines and the Formation of Individual Colonies by HEK293 and MCF-7 but not A549 in Nanofiber/Microfiber Scaffolds:

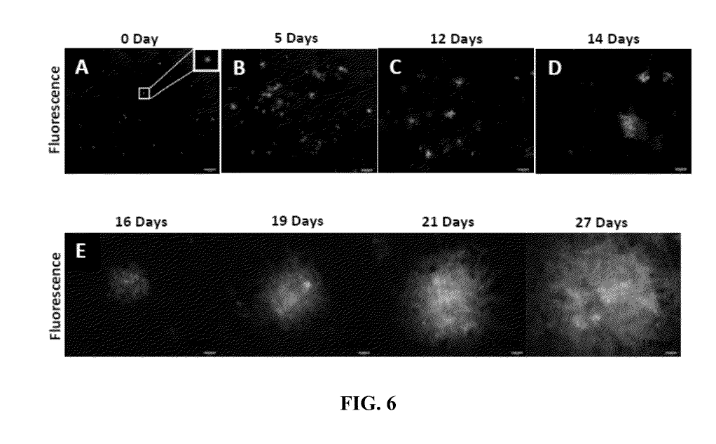

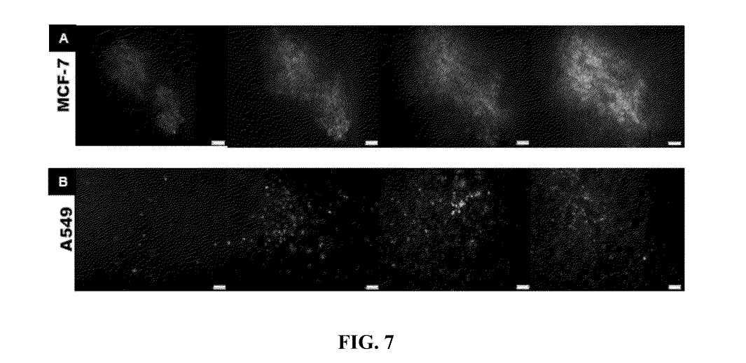

In order to monitor cell growth over a long time, GFP-HEK293 cells were seeded at a low density of 5.8×10³ cells/cm² in PLA/PET scaffold comprised of 90% PET microfibers (avg. diameter 13 μm) and 10% PLA nanofibers (avg. diameter ˜700 nm). The formation of small colonies from individual cells or a cluster of several cells was observed within the scaffold for two weeks (FIG. 6 A-D). The growth of four well-separated colonies was monitored for extra 14 days and the expansion of one of them is illustrated in FIG. 6 E. The time-dependent growth of GFP-MCF-7 and GFP-A549 cells was also investigated in CA/Rayon scaffold comprised of 90% Rayon microfibers (avg. diameter 9 μm) and 10% CA nanofibers (avg. diameter ˜400 nm). Cells were seeded at a low density of (2.5×10³ cells/cm²) and cultured for 40 days. MCF-7 cells grow into colonies in time-dependent manner. The growth of two adjacent MCF-7 colonies was monitored until fused together and formed one large colony (FIG. 7 A). In contrast to HEK293 and MCF-7 cells, A549 cells grow and scatter throughout the scaffold and remain as single cells (FIG. 7 B).

Testing the Efficiency of Growth of Various Cell Lines in the Nanofiber/Microfiber Scaffolds:

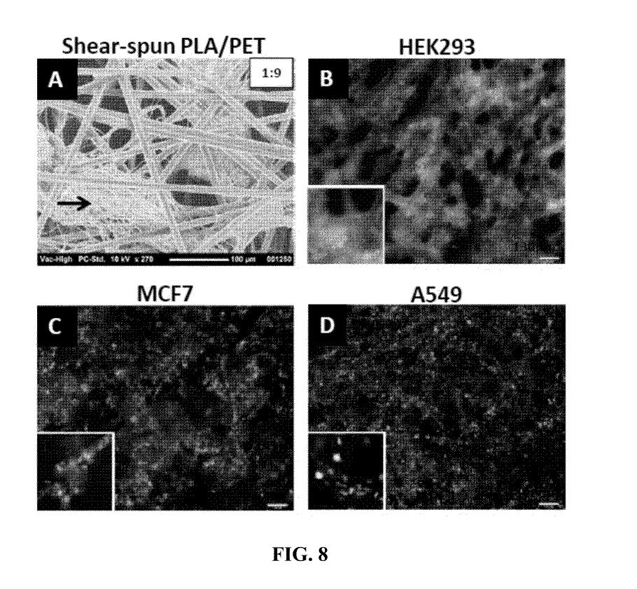

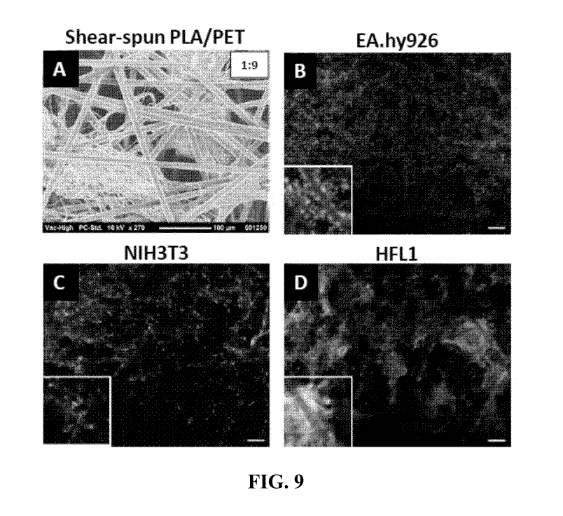

To test the feasibility of culturing various cell types within the nanofiber/microfiber scaffolds, a wide range of established cell lines were chosen, including mouse embryonic fibroblasts (NIH3T3), human foetal lung fibroblasts (HFL1), human umbilical vein endothelial cells (EA.hy926), human lung cancer (A549), human breast cancer (MCF-7) and human embryonic kidney (HEK293) cells. Tested cells were seeded at a density of 3.3×10⁴ cells/cm² and cultured in the appropriate medium for 20 days. All cultured cell types grew efficiently in the PLA/PET scaffold comprised of 90% PET microfibers (avg. diameter 13 μm) and 10% PLA nanofibers (avg. diameter ˜700 nm) (FIGS. 8 and 9). Notably, cells exhibited a distinct pattern of cell growth. For example, A549 cells showed a highly migrating pattern of growth and didn't form large 3D clusters, while MCF-7 and HEK293 cells formed compact colonies or large clusters of cells. HFL1 and NIH3T3 fibroblasts showed a tendency to grow in long tangles along the microfibers, while EA.hy926 cells formed finger-like extensions.

Determining the Optimal Nanofiber:Microfiber Ratio for Efficient Attachment of Cells to the Scaffolds:

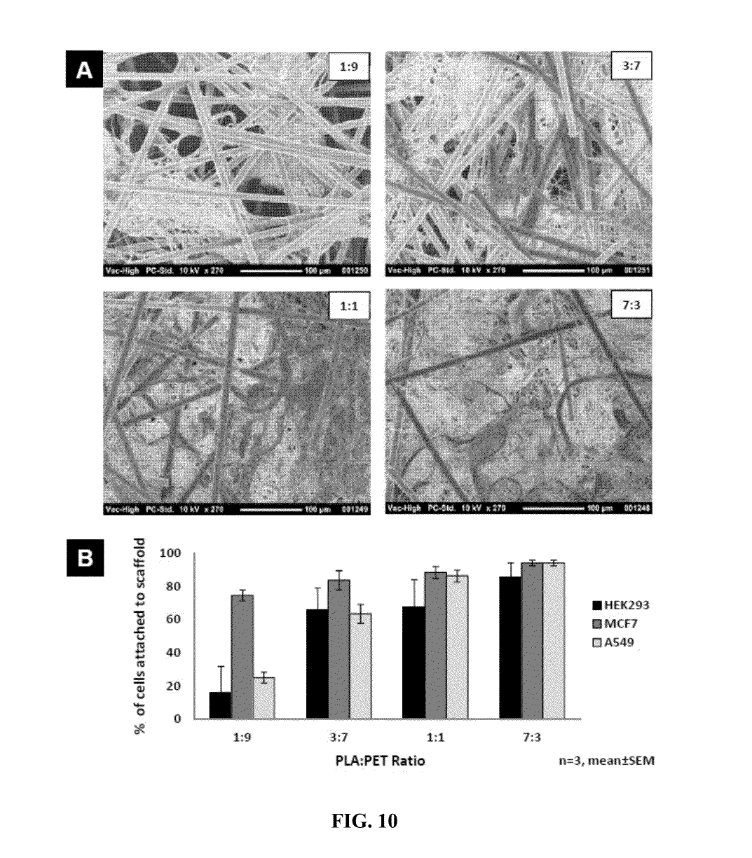

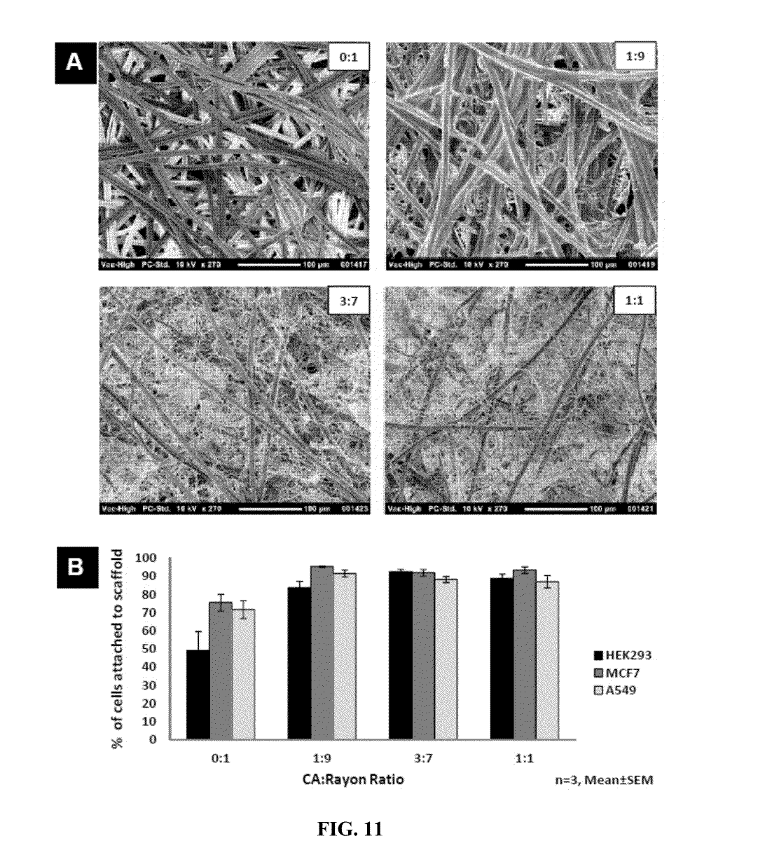

GFP-HEK293, GFP-MCF-7 and GFP-A549 cell lines were seeded at a high density of 1.6×10⁵ cells/cm² in a panel of PLA:PET scaffolds containing different nanofiber:microfiber ratios (FIG. 10 A). The outcome clearly demonstrates that the increase in the nanofiber:microfiber ratio in the PLA/PET scaffolds results in better attachment of GFP-HEK293 and GFP-A549 cells to the scaffold (FIG. 10B). This effect was less obvious for GFP-MCF-7 cells which show very efficient attachment to the scaffold with the lowest PLA:PET ratio. All tested cell lines attach efficiently to the PLA/PET scaffold with the highest content of nanofibers (7:3 ratio), but they grow mainly on the surface and don't penetrate the scaffold. Therefore, PLA/PET scaffold should consist of nanofiber:microfiber ratio between 3:7 and 1:1 for optimal cell attachment. Testing the growth of generated GFP-expressing cell lines in a panel of nanofiber/microfiber scaffolds showed the suitability of the CA/Rayon scaffold for 3D culturing of mammalian cells. In contrast to the PET microfiber scaffold, GFP-HEK293, GFP-MCF-7 and GFP-A549 cells were able to attach and grow effectively in the Rayon microfiber scaffold, while the addition of only 10% CA nanofibers to the scaffold (CA:Rayon, 1:9 ratio) maximizes cell attachment efficiency (FIG. 11B).

The Morphology of 3D Cell Culture Inside the Scaffold:

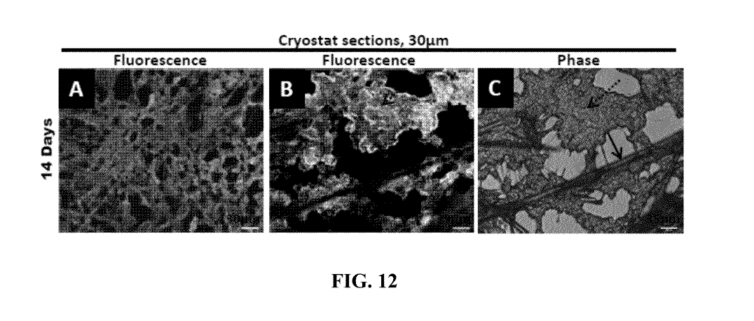

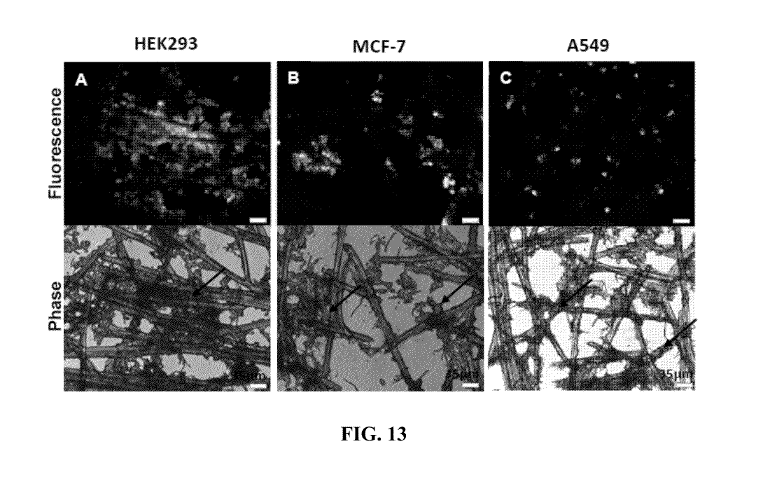

A protocol was developed for preparing cryosections of the scaffold with cultured mammalian cells. For preparing cryosections, generated GFP-expressing HEK293 cell line was seeded at a high density (3.3×10⁵ cells/cm²) in the PLA/PET scaffold and cultured for 14 days. The scaffold with cells was fixed with 4% paraformaldehyde (PFA), embedded in the OCT compound and then frozen at 20° C. Leica CM1850 cryostat was used to prepare 30 μm sections of embedded scaffolds. Fluorescent and phase contrast images of GFP-HEK293 cells grown in the PLA/PET scaffold reveal clusters of cells, which grow preferentially in cross junctions of microfibers, where nanofibers are mainly located (FIG. 12). The morphology of 3D cell culture was further investigated in CA/Rayon scaffold using 3 different cancer cell lines HEK293, MCF-7 and A549 (FIG. 13). Tested cells were seeded at a density of 1.25×10⁴ cells/cm² (4.0×10³ in 96-well plate) and cultured for 14 days. Cryostat sections reveal that HEK293 and MCF-7 cells grow into 3D cell clusters mainly at the microfibers cross-junctions, whereas A549 cells remain as single cell culture.

Analysis of the Viability of Cultured Mammalian Cells in the Scaffold:

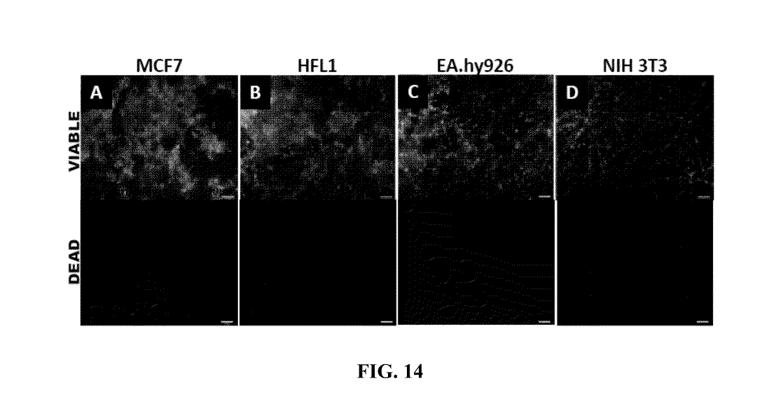

The LIVE/DEAD viability/cytotoxicity Kit (Invitrogen) was used to test the viability of a panel of cell lines cultured in various scaffolds. FIG. 14 demonstrates cell viability of four tested cell types, MCF-7, HFL1, EA.hy926 and NIH3T3, cultured in PLA/PET (1:9 ratio) scaffolds. Cells were seeded at a density of 3.3×10⁴ cells/cm² and cultured for 20 days. To detect viable and dead cells, the scaffolds with cultured cells were incubated with 1 μM Calcein-AM and 2 μM Ethidium homodimer-1 (EthD-1). Green fluorescence reveals viable cells due to hydrolysis of Calcein-AM, whereas dead cells fluoresce red due to binding of EthD-1 to DNA. This analysis indicates that culturing tested cell lines in the PLA/PET scaffold results in viable cell clusters, containing very few dead cells.

Analysis of Cell Infiltration and Scaffold Integrity:

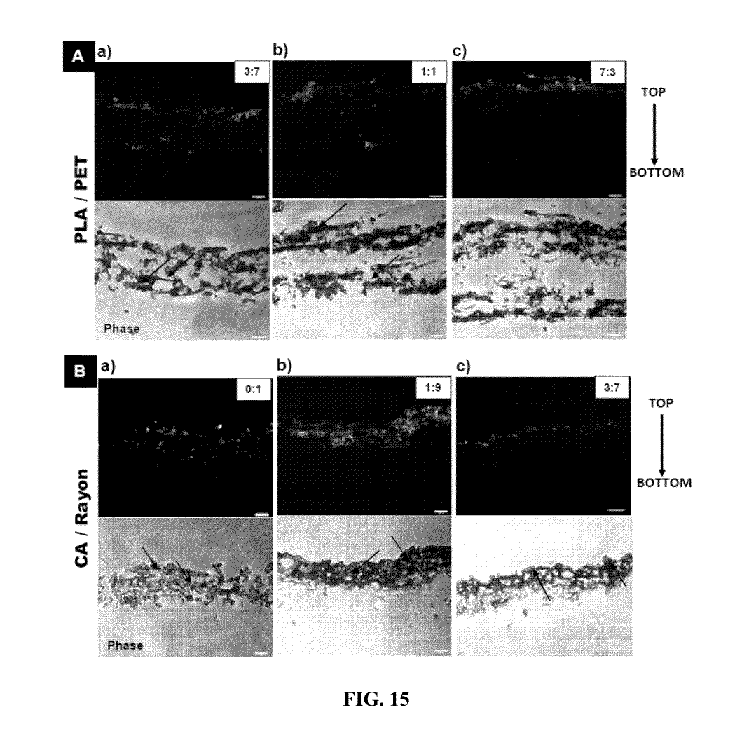

To investigate cell infiltration through PLA/PET and CA/Rayon scaffolds, high cell number (1.0×10⁵ in 96-well plate) of GFP-HEK293 cells were seeded in scaffolds with varying nanofiber:microfiber ratios. After 2 days of culture, the scaffolds were washed with PBS and fixed with 4% PFA. The cross-section analysis reveal that cells can infiltrate and grow inside CA/Rayon and PLA/PET scaffolds up to 1:9 and 1:1 nanofiber:microfiber ratio respectively (FIG. 15 A a-b and B a-b). However, at higher nanofiber:microfiber ratios cells grow on the top of the scaffold (FIG. 15 A c and B c) because nanofiber density becomes high that covers the pores between the microfiber mesh and as a result prevents cell infiltration. Additionally, the cryosection analysis shows the intact and well-integrated structure of CA/Rayon scaffolds in comparison to the loose and disintegrated structure of PLA/PET scaffolds. The intactness and integrity of the scaffold can influence the 3D cell growth and morphology. This means that cellulose acetate and rayon biomaterials form a scaffold that is more suitable for histological investigations than PLA and PET.

Comparison of Cell Proliferation Rate Between 2D Tissue Culture Plates and 3D Scaffolds:

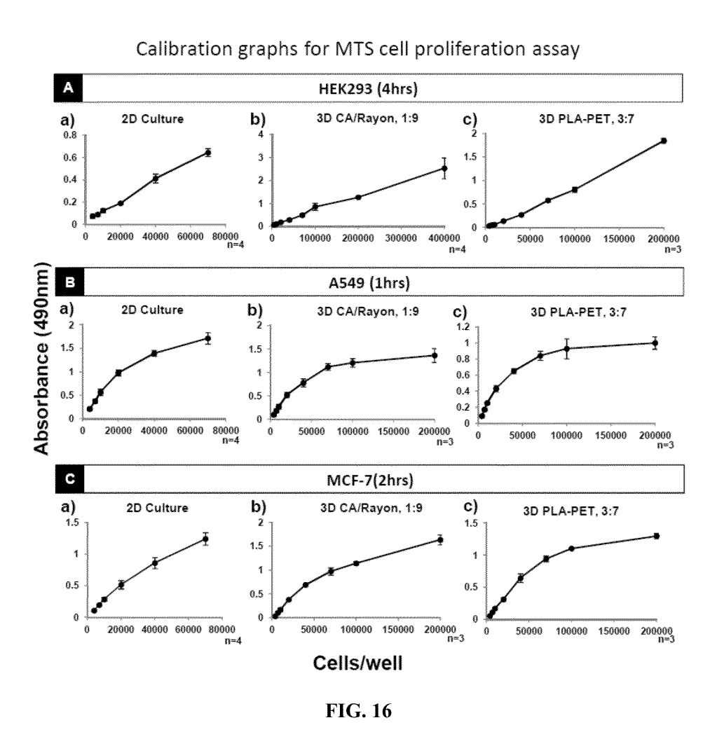

Cell proliferation was measured using the MTS CellTiter 96® Aqueous One Solution assay (Promega), in which the quantity of formazan product as measured by the absorbance at 490 nm is directly proportional to the number of viable cells in culture. Because cells metabolize MTS solution at different rate in 2D cultures and 3D scaffolds, cell proliferation cannot be compared between the two different models using the absorbance values. Thus, calibration graphs were generated and used to convert the absorbance values to cell number in order to be able to compare cell proliferation data between 2D and 3D models, provided that the same incubation time is applied (FIG. 16). The graphs show the linear correlation between the absorbance and known cell number which clearly does not hold at high cell number of A549 and MCF-7 cells. To compare the cell proliferation the same cell number (4.0×10³ in 96-well plate) of HEK293, A549 and MCF-7 cell lines are seeded in 2D culture plates and 3D nanofiber/microfiber scaffolds (PLA/PET or CA/Rayon). Cell proliferation is measured over the course of 8 and 10 days. The cell proliferation analysis demonstrates that the structural topography of the nanofiber/microfiber scaffolds affect differently the cell proliferation rate of cancer cell lines when compared to tissue culture plates, depending on the cell type characteristics. HEK293 cells proliferate slightly slower in 3D scaffolds but there is no significant difference in cell number. The cell proliferation rate of the lung cancer A549 cell line is significantly higher in 2D TCP than in 3D scaffolds (* p<0.05). Lastly, in the first 4-6 days the cell proliferation rate of the breast cancer MCF-7 cell line in 3D scaffolds was the same as in 2D TCP. However, at day 8 the cell proliferation increased significantly in the 3D scaffold than in 2D TCP and cell number has doubled (** p<0.005).

Chemosensitivity of Cancer Cell Lines to Anticancer Drugs in 2D Tissue Culture Plates and 3D Scaffolds:

25000 cells of tested cell lines were seeded in 2D TCP and 3D scaffold. Cells were treated with anticancer drugs for 48 hrs and then cell viability was measured using MTS CellTiter 96® Aqueous One Solution assay (Promega). The analysis of chemosensitivity reveals the significant difference in cancer cell response to anticancer drugs when comparing 3D scaffold with 2D cell cultures. Furthermore, the cancer cell lines A549 and MCF-7 show the opposite drug response in both cell model. Lung cancer A549 cells were more sensitive to 5-fluorouracil and doxorubicin in 2D TCP than in 3D scaffolds (A) whereas breast cancer MCF-7 cells were more chemosensitive in 3D scaffold than in 2D TCP.

Generation of Stable Cell Lines Expressing RFP or BFP:

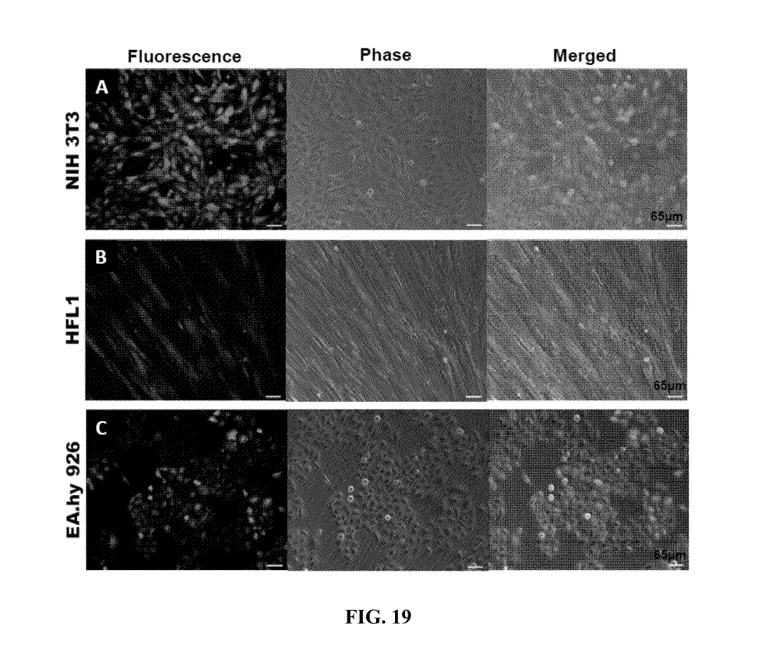

A panel of stable cell lines expressing RFP or BFP is generated in order to visualize and differentiate between different cell types in co- and tri-cultures. The generation of stable cell lines is achieved using the lentiviral protein expression system. Mouse embryonic fibroblast (NIH3T3), human foetal lung fibroblast (HFL1) and human umbilical vein endothelial (EA.hy926) cell lines were infected with recombinant lentiviruses to direct the expression of red or blue fluorescent protein (RFP or BFP). Stably transduced cells were selected with 1.0 μg/mL puromycin for 2-3 weeks. FIG. 19A-C shows fluorescent and phase contrast images of NIH3T3 (A), HFL1 (B) and EA.hy926 (C) with stable expression of RFP. Notably, selected cell lines exhibit different patterns of growth when cultured in 2D tissue culture plates.

The Growth Pattern of Co- and Tri-Cultures in Nanofiber/Microfiber Scaffold:

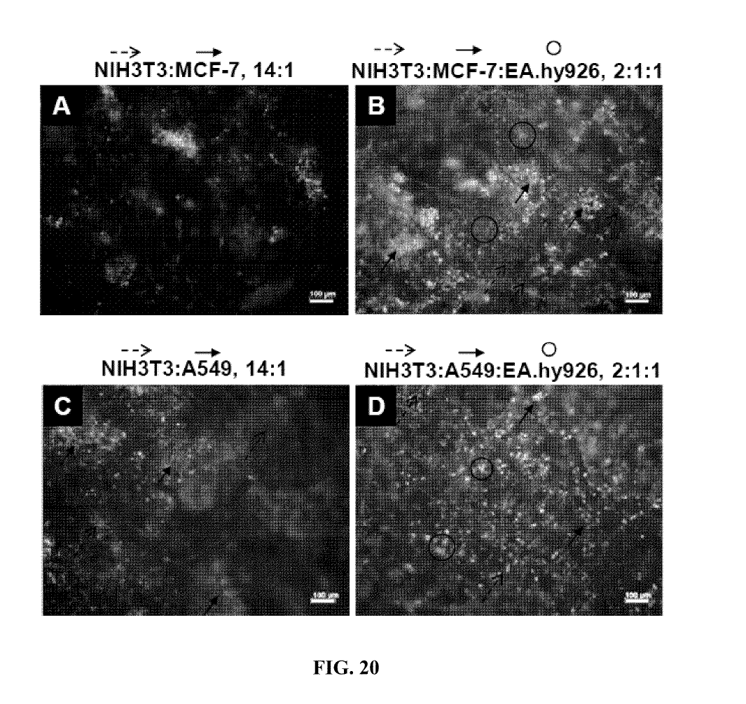

Co- and tri-cultures were performed between GFP-cancer cell lines (MCF-7 or A549), RFP-fibroblast (NIH3T3) and/or BFP endothelial cells (EA.hy926). The seeding cell number ratio between the different cell types is indicated in the figures. The fluorescence microscopy and confocal z-stack images demonstrate the pattern and the efficient growth of different cell types when co-cultured or tri-cultured in CA/Rayon, 1:9 scaffold. Same as in monoculture, MCF-7 cells formed small 3D cell clusters and colonies in co- and tri-cultures (FIGS. 20 A-B and 21 A-D). Moreover, MCF-7 colonies appear to be more compact when fibroblast:cancer ratio has increased to 14:1 (FIG. 20 A). A549 cells maintained the scattering growth pattern in the tri-culture (FIG. 20 D). However, when fibroblast:cancer cell seeding ratio becomes high (14:1), A549 cells seem to be sequestered in localized regions (FIG. 20 C).

1. A method of culturing cells and/or controlling cell behaviour, which method comprises:
(a) introducing one or more cells into a medium; and
(b) allowing said one or more cells to grow within and/or interact with the medium,
wherein said medium comprises a three dimensional homogeneous distribution of shear-spun nanofibers.

2. The method of claim 1, which is a method of culturing cells and/or measuring cell proliferation.

3. The method of claim 2, which is a method of culturing mammalian cells.

4. The method of claim 1, which is a method of regulating or promoting cell function in said one or more cells, or regulating or promoting gene expression in said one or more cells.

5. The method of claim 1, wherein said medium is a solid scaffold made by:
(a) wet laying a mixture of microfibers and said nanofibers, to form a nonwoven material;
(b) wet laying said nanofibers onto a woven or nonwoven material comprising microfibers; or
(c) wet laying said nanofibers onto themselves to form a nonwoven material.

6. The method of claim 1, wherein said nanofibers comprise nanofibers of one or more polymers selected from the group consisting of a polyolefin, a polystyrene, a polycaprolactone, a polyacrylonitrile, a polyvinylidenedifluoride, a poly(vinyl chloride), a polytetrafluoroethylene, a poly(α-methylstyrene), a poly(acrylic acid), a poly(isobutylene), a poly(acrylonitrile), a poly(methacrylic acid), a poly(methyl methacrylate), a poly(l-pentene), a poly(1,3-butadiene), a poly(vinyl acetate), a poly(2-vinyl pyridine), a 1,4-polyisoprene, a 3,4-polychloroprene, a poly(ethylene oxide), a polyformaldehyde, a polyacetaldehyde, a poly(3-propionate), a poly(10-decanoate), a poly(ethylene terephthalate), a polycaprolactam, a poly(11-undecanoamide), a poly(hexamethylenesebacamide), a poly(m-phenylene terephthalate), a poly(tetramethylene-m-benzenesulfonamide), a polyacetal, a polyetheretherketone, a polyetherimide, a polyamide, a polyurea, a polyamideimide, a polyarylate, a polybenzimidazole, a polyester, a polycarbonate, a polyurethane, a polyimide, a polyhydrazide, a phenolic resin, a polysilane, a polysiloxane, a polycarbodiimide, a polyimine, an azo polymer, a polysulfide, and a polysulfone.

7. The method of claim 1, wherein said nanofibers comprise nanofibers of one or more natural materials selected from the group consisting of cellulose, cellulose acetate, PLA, silk, chitin, hemp and cotton.

8. The method of claim 1, wherein said nanofibers have an average length of from 0.02 to 2000 mm.

9. The method of claim 1, wherein said nanofibers have a length to diameter (L:D) aspect ratio of from 20:1 to 200,000:1.

10. The method of claim 1, wherein said nanofibers have an average diameter of from 100 nm to 10 μm.

11. The method of claim 1, wherein said medium comprises a three dimensional homogeneous distribution of microfibers and said nanofibers.

12. The method of claim 11, wherein said medium is a solid scaffold made by:
(a) wet laying a mixture of said microfibers and nanofibers to form a nonwoven material, or
(b) wet laying said nanofibers onto a woven or nonwoven material comprising said microfibers.

13. The method of claim 11, wherein said solid scaffold is a disc with a thickness of 100 μm or more.

14. The method of claim 11, wherein said microfibers comprise microfibers of one or more selected from the group consisting of cotton, cellulose, Lyocell, acetate, cellulose acetate, rayon, silk, wool, hemp, spandex, polyolefin, polyamide, aramid, acrylic, polyester, polyurethane, glass microfibers, and fibreglass.

15. The method of claim 11, wherein the said nanofibers account for at least 1% by weight of the total weight of said nanofibers and said microfibers.

16. The method of claim 11, wherein said scaffold has pores of size 2 μm to 500 μm.

17. The method of claim 11, wherein said microfibers are microfibers of polyethylene terephthalate (PET), and said nanofibers are nanofibers of polylactic acid (PLA), and the weight ratio of said fibers of PET to said fibers of PLA is from 7:3 to 1:1.

18. The method of claim 11, wherein said microfibers are microfibers of rayon, and said nanofibers are nanofibers of cellulose acetate (CA), and the weight ratio of said fibers of rayon to fibers of CA is from 9:1 to 7:3.

19. The method of claim 11 for use in identifying the optimal nanofiber:microfiber ratio for efficient attachment of a given cell.

20. The method of claim 1, wherein said medium comprises one or more further substances selected from the group consisting of a growth factor, anti-oxidant, differentiation inducer, hormone, vitamin, nucleic acid, drug, humectant, emollient, peptide, conditioner and cosmetic.

21. The method of claim 1, wherein said medium is a dispersion of said nanofibers in at least one liquid selected from the group consisting of cell culture media, PBS, water and other biologically compatible liquids.

22. The method of claim 21, wherein the medium comprises 0.1% to 10% by weight of said nanofibers.

23. A medium comprising a three dimensional homogeneous distribution of shear-spun nanofibers, which medium is suitable for use in culturing cells and/or controlling cell behaviour in a method as defined in claim 1.

24. The medium of claim 23, which medium is a solid scaffold comprising a three dimensional homogeneous distribution of microfibers and said nanofibers.

25. The solid scaffold of claim 24, said scaffold being made by:
(a) wet laying a mixture of said microfibers and said nanofibers to form a nonwoven material; or
(b) wet laying said nanofibers onto a woven or nonwoven material comprising said microfibers.

26. The solid scaffold of claim 25, which is a disc with a thickness of 100 μm or more.

27. The medium of claim 23, which medium is a dispersion of said nanofibers in a liquid.

28. The dispersion of claim 27, wherein said liquid is selected from the group consisting of cell culture media, PBS, water and other biologically compatible liquids.

29. A process of producing a medium as defined in claim 23, which process comprises:
(a) wet laying a mixture of microfibers and said nanofibers, to form a nonwoven material;
(b) wet laying said nanofibers onto a woven or nonwoven material comprising microfibers; or
(c) wet laying said nanofibers onto themselves to form a nonwoven material.

30. The medium of claim 23, which comprises cultured cells.

31. A method of measuring cell proliferation, cytotoxicity and/or chemosensitivity in a sample of the medium comprising cultured cells as defined in claim 30.
